(19) United States
(12) Patent Application Publication
(10) Pub. No.: US 2022/0167645 A1
(43) Pub. Date: Jun. 2, 2022

(54) METHODS OF IMPROVING FISH FEED CONVERSION RATE AND SURVIVAL

(71) Applicant: AQUINOVO LTD., Ness-Ziona (IL)

(72) Inventors: Limor ZIV, Mazkeret Batya (IL); Nissim CHEN, Givataym (IL); Minh Anh PHAM, Ho Chi Minh City (VN)

(21) Appl. No.: 17/437,216

(22) PCT Filed: Mar. 11, 2020
(86) PCT No.: PCT/IL2020/050283

§ 371 Date: Sep. 8, 2021

Related U.S. Application Data
(60) Provisional application No. 62/816,225, filed on Mar. 11, 2019.

(51) Int. Cl. A23K 20/147; A23K 50/80; A23K 40/30

(52) U.S. Cl. CPC A23K 20/147; A23K 40/30; A23K 50/80

(57) ABSTRACT

The present invention relates to methods for improving fish feed conversion rates, health and survival by tachykinin antagonists. Specific fish feed consumptions comprising tachykinin antagonists are also provided.

### FIELD OF THE INVENTION

The present invention relates to fish feed consumption and specifically relates to methods of improving fish feed conversion rates, health and survival by tachykinin antagonists.

### BACKGROUND OF THE INVENTION

Tachykinins (TKs, also termed neurokinins, NKs) are a group of short neuropeptides (7-11 amino acids) that share the common carboxy-terminal amino acid sequence Phe-X-Gly-Leu-Met-NH₂ (where X is an aromatic residue or a beta-branched aliphatic residue), (Steinhoff et al. 2014. Physiological Reviews 94(1): 265-301). The TKs are the largest group of peptides in the world whose members are present in all animal species, from lower invertebrates to mammals. The major target of TKs is the central and peripheral nervous system where they act as neurotransmitters/neuromodulators. As such, TK are released from nerve terminals to synapse, and by binding to their receptors, stimulate contraction of smooth muscles, mainly in the gastrointestinal (GI) tract. TKs are also involved in pain processing, inflammation, stress and anxiety behaviors, emesis, fluid secretion in intestinal epithelium, and gonad function.

The biological activity of TKs depends on their interaction with three distinct G protein-coupled neurokinin receptors NK1R, NK2R, and NK3R (Steinhoff et al. 2014 ibid). The three receptors share considerable structural homology, reflecting their common mechanism of action. Consistent with their physiological roles, NK receptors are densely scattered in the nervous system and gastrointestinal tract, but also have sparse and irregular expression in immune cells, respiratory system, urogenital tract, gonads and skin. The three classes of tachykinin receptors have preferential binding affinities for SP, NKA, and NKB, respectively. However, each neurokinin receptor has at least moderate affinity for each neurokinin ligand (Leffler et al. 2009. Biochemical Pharmacology 77(9): 1522-30). Therefore, NK receptors can be fully activated by any tachykinins, when provided in sufficient amount.

Hundreds of NKR antagonists that differ in structure (peptides or small molecule), NKR selectivity and species specificity are known at present (Hoogerwerf and Sarna 2006. Digestive Diseases 24(1-2): 83-90; Misu et al. 2013. Bioorganic and Medicinal Chemistry 21(8): 2413-17). Antagonists can interact with the same sites as the endogenous agonists and shut down receptor signaling (orthosteric interaction), or bind to different sites of the receptor, and attenuate endogenous TK signaling (allosteric interaction). In addition, interspecies variation in small number of amino acid residues of the NK receptors may influence interspecies divergence in affinity for specific antagonists.

Several peptidic and non-peptidic NKB antagonists are known in mammals. G. Drapeau et al., (Regul. Peptides, 31: 125-135, 1990) discloses the compound SR142801 (Trp⁷, β-Ala⁸-Neurokinin A, 4-10) as a potent antagonist of the tachykinin NK3 receptor in mammalians. O'Harte, F. (J. Neurochem. 57(6): 2086-2091, 1991) discloses the peptide analog denoted Ranakinin, an NK1 tachykinin receptor agonist isolated with neurokinin B from the brain of the frog Rana ridibunda. SB-222200 (Sarau et al., 2000, J Pharmacol Exp Ther 295:373-381); Osanetant (SR-142,801) and talnetant (SB 223412) (Saran et al., 1997, J Pharmacol Exp Ther 281:1303-1311).

WO 2013/018097 discloses NKB and NKF agonists in fish useful in advancing the onset of puberty, regulating the timing and amount of ovulation and spawning, synchronization or stimulation of reproduction, enhancing the development of gammets, enhancing vitellogenesis, induction of GnRH, induction of Kisspeptine, increase in the levels of hypothalamic neurohormones, increasing the level of LH or FSH and induction of oocyte maturation.

WO 2016/009439 discloses peptide-based NKB antagonists and their use in inhibiting or delaying fish maturation of reproductive system.

The effect of TKs on smooth muscle contraction and gut motility is a fundamental observation underlying TKs and NKRs physiological role (Maggi et al. 1990. British Journal of Pharmacology 101(4): 996-1000). The major sources of TK neuropeptides in the gastrointestinal tract are enteric neurons followed by nerve fibers from dorsal root and vagal ganglia. TK-releasing nerve fibers are in close proximity to cells expressing NKRs. The physiological roles of TK in the GI tract are conserved between vertebrates and are governed by all NK receptors with some inter-species differences in distribution and ligand affinities (Severini et al. 2002. Pharmacological Reviews 54(2): 285-322). Aside contraction, TKs are involved in the regulation of epithelial fluid secretion during digestion and pathogen insult, inflammation and pain.

Gut motility plays an essential role in the breakdown and transport and digestion of food in the gastrointestinal (GI) tract and is achieved by contraction and relaxation of smooth muscles located in the gut wall. Food is transported from the mouth, where digestion starts, along the stomach and intestine where it is further digested, before nutrients are eventually absorbed. To facilitate digestion, the ingested food is mixed so that enzymes and acid can gain access to all food particles. The efficiency of food digestibility is predicted by measurement of retention time of food chemical composition in the GI tract. High retention time may result in decrease in food uptake, while fast clearance of the GI reduces digestibility of organic matter. Thus, regulation of gut motility is an important factor for optimizing animal feed consumption and growth.

Gut motility regulation is a complex system in which central and local neural inputs work in concert to produce synchronized and adapted contraction and relaxation waves. TKs are strong stimulants of gut motility in all vertebrates acting on both central and local levels. TKs and NKRs can regulate gut motility of isolated gut segments in all vertebrates including fish. Substance P and neurokinin A (NKA) were shown to induce or enhance contraction of isolated intestine strips of many fish species: thorny skate, South American lungfish, Gray Bichir and Rainbow Trout (Gra\ddot{ }ns A. and Olsson C. (2011) Gut Motility. In: Farrell A. P., (ed.), Encyclopedia of Fish Physiology: From Genome to Environment, volume 2, pp. 1292-1300.San Diego: Academic Press). Furthermore, NKA administration to zebrafish larvae in vivo increased gut motility as determined by live imaging of the gut (Holmberg 2004).

Tachykinins stimulate electrolyte and fluid secretion from the intestinal epithelium by activating NKR receptors. Fluid secretion in the gut facilitates propulsion, digestion and mediates protective secretory responses to infection (Holzer 2004. Handbook of Experimental Pharmacology 164: 511-58). In some mammals, NKRs also involved in gastric secretion of acid and pepsinogen and intestinal secretion of VIP, glucagon-like peptide-1, and somatostatin.

The involvement of TKs in membrane filtration during GI inflammation is well documented. TKs are vigorous pro-inflammatory agents in the gut thus, their inhibition can mitigate the adverse effects of inflammation on gut function. It was shown that mice genetically deficient in the NK1R are protected from the secretory and inflammatory changes as well as from epithelial cell damage induced by Clostridium difficile, toxin A (Castagliuolo et al. 1998. Journal of Clinical Investigation 101(8): 1547-50).

The gastrointestinal tract is also responsible for up to 70% of the animal's immune response activity. Once bacteria, pathogens or their toxins pass through the protective epithelial layer of the gastrointestinal tract lining, the immune system triggers an immune response. While an appropriate inflammatory response is necessary, excessive or prolonged inflammation can become detrimental to the animal. Immune system activation consumes significant amounts of nutrients. Thus, excessive immune response may pull nutrients and energy away from other key functions within the animal such as growth and reproduction, and also can prevent successful immune response against future insult.

TK antagonists are clinically tested in human for treating gastrointestinal disorders, such as irritable bowel syndrome (IBS), inflammatory bowel disease (IBD), chronic diarrhea, gastroparesis, nausea and vomiting (Hoogerwerf and Sarna 2006 ibid). The FDA approved drug Aprepitant (Emend® Merck), is an NK1 receptor antagonist effective in the prevention of chemotherapy-induced acute or delayed nausea and vomiting.

Neurokinin B-NK3R signaling plays an important role in controlling mammalian reproduction. Inactivating mutations in the tachykinin 3 (tac3) or tac3 receptor (NKBR) gene are associated with pubertal failure and congenital hypogonadotrophic hypogonadism in humans, suggesting that NKB may have a critical role in human reproduction. In fish, NKB is involved in HPG axis regulation, mainly displaying autocrine/paracrine actions within the fish pituitary (J. Biran et al. 2012. PNAS 109:10269-74; Jakob Biran et al. 2014. Endocrinology 155(12): 4831-42; Chen et al. 2018. General and Comparative Endocrinology 260: 125-35; Jin et al. 2016. Development & Reproduction 20(1): 51-61; Ogawa et al. 2012. Journal of Comparative Neurology 520(13): 2991-3012; Zmora et al. 2017. Journal of Endocrinology 233(2): 159-74).

To date, a large number of natural TKs have been identified in a wide range of species from invertebrates to mammals. In fish, Tac1 gene encodes the TKs substance P (SP) and NKA through alternative splicing. Tac3a/b genes produce the TK peptides NKBa/b and NKFa/b respectively and Tac4a/b encodes peptides EKA_{1/2} and EKB_{1/2} respectively (Hu et al. 2014. General and Comparative Endocrinology 208: 94-108). Tachykinins and their receptors were identified in many fish species (Biran 2012, PNAS 109:10269-10274 ibid). High identity was found among different fish species in the region encoding the NKB; all shared the common C-terminal sequence. Although the piscine Tac3 gene encodes for two putative tachykinin peptides, the mammalian orthologue encodes for only one. The second fish putative peptide, referred to as neurokinin F (NKF), is unique and found to be conserved among all tested fish species.

It was suggested that the NKB/NKBR system may participate in neuroendocrine control of fish reproduction. Biran et al., (2014, Endocrinology 155, 4831-42) have showed that NKB and NKF, secreted by the fish brain, can stimulate the release of follicle stimulating factor (FSH) and LH by direct (through activation of specific receptors at the pituitary level) or indirect (through the brain) mechanisms.

The three NK receptors are also expressed throughout the central nervous system including forebrain and brainstem regions which are involved in stress, anxiety and motivated behaviors. Several mammalian models have showed elevated brain TK during stress episodes and that NKR antagonist can block anxiogenic and depressive behaviors (Steinhoff et al. 2014 ibid). NK1R antagonist MK869 had antidepressant effect in patients with moderate to severe depression (Kramer M S, et al. 1998. Science 281: 1640-1645).

Fish is an important source of protein and other nutrients for humans. Over 32,000 species of fish have been described, making them the most diverse group of vertebrates. However, only a small number of species are consumed as food in virtually all regions around the world. While previous publications disclosed fish TK and NKB agonists and antagonists, for enhancing or delaying maturation by intervening the reproductive system, none of the prior publications disclose the use of fish TK antagonists for improvement of feed conversion rate (FCR) and protecting from stress-induced mortality.

### SUMMARY OF THE INVENTION

The present invention is based on the finding that inhibition of tachykinin (TK) receptor activity in fish by TK antagonists results in improvement of feed conversion rate (FCR) and overall fish health and survival.

It was surprisingly found that TK antagonists have the capacity to improve digestibility and to increase availability of nutrient and energy in favor of fish somatic growth processes for a given amount of feed, even without affecting the reproductive system. The lower feed conversion rates achieved following administration of TK antagonists, improves fish productivity and profitability.

TKs are involved in many aspects of gut physiology. The contribution of TK antagonists to digestibility and their use to improve the rate by which food converts into fish substance (feed conversion rate), is herein suggested for the first time. Experimental observations provided herein show that fish FCR and survival are significantly improved following oral consumption of TK antagonists. It is further suggested that TK antagonist treatment inhibits excessive inflammation response in the fish gut thereby improving the ability of fish to process food and that TK antagonist treatment reduces excessive inflammatory response, in the soma and skin, leading to increased resistance to pathogens and improves overall health and survival.

The present invention provides, according to one aspect, a method of improving fish feed conversion rate (FCR), or at least one fish health parameter selected from the group consisting of: survival, skin condition and resistance to pathogens, the method comprising administering to a fish population a composition comprising at least one tachykinin (TK) antagonist.

According to some aspects, a method of improving fish feed conversion rate (FCR) is provided, the method comprises administering to a fish population a composition comprising at least one tachykinin (TK) antagonist.

According to certain aspects, the present invention provides a method for improving at least one fish health parameter selected from the group consisting of: survival, skin condition and resistance to pathogens, the method comprises administering to a fish population a composition comprising at least one tachykinin (TK) antagonist.

Any TK antagonist compound capable of binding to at least one piscine TK receptor and inhibiting its activity may be used according to the present invention in methods and compositions for improving feed conversion rate and/or at least one fish health parameter.

According to some embodiments, the improved fish health parameter is selected from the group consisting of: survival, skin condition and resistance to pathogens.

According to some embodiments, the health parameter improved is survival.

According to some specific embodiments, skin health or condition is improved.

According to other embodiments, the fish population fed with the compositions of the present invention comprises fish aged 3-1200 dph.

According to some embodiments, the fish population fed with the compositions of the present invention comprises fish aged 5-1000 dph.

According to some embodiments, the fish population fed with the compositions of the present invention comprises fish aged 25-250 dph.

According to some embodiments, the fish population fed with the compositions of the present invention comprises majority of fish aged 10-150 dph.

According to some embodiments, the tilapia fish population fed with the compositions of the present invention comprises majority of fish aged 5-150 days, namely up to about five months.

According to some embodiments, the tilapia fish population fed with the compositions of the present invention comprises majority of fish aged 10-90 days, 30-130 days or 30-90 days. Each possibility represents a separate embodiment of the present invention.

According to some embodiments, the salmon fish population fed with the compositions of the present invention comprises majority of fish aged 10-1200 days, namely up to about 3 years.

According to some embodiments, the fish are edible fish.

According to other embodiments, the fish are ornamental fish.

According to yet other embodiments, the fish population comprises a majority of adult fish, namely sexually mature fish.

According to yet other embodiments, the fish population comprises a majority of juvenile fish, namely non-sexually mature fish

According to some embodiments, the treated fish population comprises a majority of male fish.

According to some embodiments, the treated fish population consists of male fish.

According to some embodiments, the treated fish population comprises a majority of female fish

According to yet other embodiments, the method comprises administering a TK antagonist to adult, mature male fish.

According to yet other embodiments, the reproductive system (e.g. gonads) of the treated fish is not affected by the TK antagonist treatment.

According to some embodiments, expression of at least one TK receptor is altered in the gut and/or skin in response to treatment with a TK antagonist of the invention.

According to some embodiments, the method comprises administration of a composition comprising a TK antagonist selected from the group consisting of a non-peptidic molecule and a peptide-based molecule. According to certain embodiments, the TK antagonist is a non-peptidic organic molecule.

According to some embodiments, the peptide-based molecule is a peptidomimetic. According to yet some embodiments, the peptidomimetic is according to Formula I:

X₁—NMeVal-X₄-Leu-Met-Z (Formula I)

wherein:
the peptidomimetic consists of 5-10 amino acids;
X₁ is a stretch of 1-6 natural or non-natural amino acid residues and optionally an N-terminal capping moiety or modification;
NMeVal is an N-methyl-Valine residue or N-methyl-D-Valine residue;
X₄ is —NH(CH₂)ₙ—CO— wherein n is 2-6; and
Z represents the C-terminus of the peptide which may be amidated, acylated, reduced or esterified. Each possibility represents a separate embodiment of the present invention.

According to some embodiments X₁ comprises at least one aromatic amino acid residue in L or D configuration.

According to other embodiments, X₁ comprised a D-Trp residue.

According to some embodiments X₁ comprises at least one negatively charged (acidic) amino acid residue.

According to some embodiments, the C-terminus is amidated.

According to some embodiments, X₁ consists of 2 or 3 amino acids and a capped N-terminus. Each possibility represents a separate embodiment of the present invention.

According to some embodiments, X₁ consists of 2 or 3 amino acid residues comprising an aromatic residue, a negatively charged (acidic) residue and an N-terminus capping moiety. Each possibility represents a separate embodiment of the present invention.

According to some embodiments, the X₁ comprises a residue selected from an aliphatic amino acid residue and a polar, uncharged residue. Each possibility represents a separate embodiment of the present invention.

According to some embodiments, the aliphatic residue is selected from the group consisting of: Ala, Ile, Leu. Each possibility represents a separate embodiment of the present invention.

According to some embodiments, the polar, uncharged residue is selected from Ser and Thr. Each possibility represents a separate embodiment of the present invention.

According to some embodiments, X₁ comprises an aromatic residue selected from the group consisting of Phe, DPhe, Trp and DTrp; a negatively charged (acidic) residue selected from Glu and Asp; and a succinyl (Succ)N-terminus capping moiety. Each possibility represents a separate embodiment of the present invention.

According to yet other embodiments, X₁ comprises an aromatic residue selected from Phe, and DTrp; an Asp residue; a succinyl (Succ)N-terminus capping moiety, and optionally a residue selected from Ile and Ser. Each possibility represents a separate embodiment of the present invention.
According to some embodiments, the method comprises administering a composition comprising a peptidomimetic according to Formula II:

X₁—NMeVal-βAla-Leu-Met-NH₂ (Formula II)

wherein, X₁ is selected from the group consisting of: Succ-Asp-Phe; Succ-Asp-DPhe; Succ-Asp-Trp; Succ-Asp-DTrp; Succ-Asp-Ile-Phe; Succ-Asp-Ile-DPhe; Succ-Asp-Ile-Trp; Succ-Asp-Ile-DTrp; Succ-Asp-Ser-Phe; Succ-Asp-Ser-DPhe; Succ-Asp-Ser-Trp; Succ-Asp-Ser-DTrp, Succ-Glu-Phe; Succ-Glu-DPhe; Succ-Glu-Trp; Succ-Glu-DTrp; Succ-Glu-Ile-Phe; Succ-Glu-Ile-DPhe; Succ-Glu-Ile-Trp; Succ-Glu-Ile-DTrp; Succ-Glu-Ser-Phe; Succ-Glu-Ser-DPhe; Succ-Glu-Ser-Trp; and Succ-Glu-Ser-DTrp. Each possibility represents a separate embodiment of the present invention.

According to some embodiments, the method of improving fish FCR and survival comprises administering a peptidomimetic consisting of 5-10 amino acid residues comprising the sequence: NMeVal-13Ala-Leu-Met (SEQ ID NO. 7).

According to some embodiment the peptidomimetic comprises a sequence of SEQ ID NO. 7, at least one aromatic amino acid residue and at least one negatively charged amino acid residue.

According to some embodiments, the peptidomimetic comprises a sequence of SEQ ID NO. 7, at least one aromatic amino acid residue, at least one negatively charged amino acid residue and at least one residue selected from an aliphatic amino acid residue and a polar, uncharged residue. Each possibility represents a separate embodiment of the present invention.

According to some embodiments, the peptidomimetic comprises a capped N-terminus.

According to some embodiments the peptidomimetic comprises an amidated C-terminus.

According to some embodiments the peptidomimetic consist of 5, 6, 7, 8, 9 or 10 amino acid residues and an optional N-terminal capping group. Each possibility represents a separate embodiment of the present invention.

According to some embodiments, the peptidomimetic consists of 5-10 amino acid residues, an amidated C-terminus and an N-terminal capping moiety. Each possibility represents a separate embodiment of the present invention.

According to some embodiments, the peptidomimetic consists of 6-7 amino acid residues comprising the sequence of SEQ ID NO. 7, and a sequence selected from the group consisting of: Succ-Asp-Phe; Succ-Asp-DPhe; Succ-Asp-Trp; Succ-Asp-DTrp; Succ-Asp-Ile-Phe; Succ-Asp-Ile-DPhe; Succ-Asp-Ile-Trp; Succ-Asp-Ile-DTrp; Succ-Asp-Ser-Phe; Succ-Asp-Ser-DPhe; Succ-Asp-Ser-Trp; Succ-Asp-Ser-DTrp, Succ-Glu-Phe; Succ-Glu-DPhe; Succ-Glu-Trp; Succ-Glu-DTrp; Succ-Glu-Ile-Phe; Succ-Glu-Ile-DPhe; Succ-Glu-Ile-Trp; Succ-Glu-Ile-DTrp; Succ-Glu-Ser-Phe; Succ-Glu-Ser-DPhe; Succ-Glu-Ser-Trp; and Succ-Glu-Ser-DTrp.

According to some embodiments the at least one N-terminal capping moiety is a dicarboxylic acid residue. According to some embodiments the at least one N-terminal capping moiety is selected from the group consisting of: succinyl, oxalyl, malonyl, glutaryl, adipoyl, pimaloyl, suberoyl, and acetyl. Each possibility represents a separate embodiment of the present invention.

According to some embodiments, the method comprises administering to fish a composition comprising a peptidomimetic selected from the group consisting of: a peptidomimetic of Formula I, as defined above; a peptidomimetic of Formula II, as defined above; and a peptidomimetic of 5-10 amino acid residues comprising the sequence of SEQ ID NO. 7.

According to some specific embodiments the peptidomimetic is selected from the group consisting of:

(SEQ ID NO. 1, AN1)
Succ-Asp-Ile-Phe-N(Me)Val-βAla-Leu-Met-NH₂;
(SEQ ID NO. 2, AN2)
Succ-Asp-Phe-N(Me)Val-βAla-Leu-Met-NH₂;
(SEQ ID NO. 3, AN3)
Succ-Asp-Ser-Phe-N(Me)Val- βAla-Leu-Met-NH₂;
(SEQ ID NO. 4, AN4)
Succ-Asp-Ile-D-Trp-N(Me)Val- βAla-Leu-Met-NH₂;
(SEQ ID NO. 5, AN5)
Succ-Asp-D-Trp-N(Me)Val- βAla-Leu-Met-NH₂;
and
(SEQ ID NO. 6, AN6)
Succ-Asp-Ser- D-Trp -N(Me)Val-βAla-Leu-Met-NH₂;

wherein Succ denotes a succinyl.

According to some embodiments, the TK antagonist peptidomimetic, used in a method of improving fish FCR, further comprises a permeability-enhancing moiety. Any moiety known in the art to facilitate actively or passively or enhance permeability of the compound into cells may be used in the peptidomimetics according to the present invention. The permeability-enhancing moiety may be connected to any position in the peptide moiety, directly or through a spacer or linker.

According to some specific embodiments, the method of improving fish FCR and survival comprises administering to fish a composition comprising the peptidomimetic Succinyl-Asp-Ser-D-Trp—N(Me)Val-βAla-Leu-Met-NH₂ (SEQ ID NO. 6, AN6).

According to some embodiments, the method of improving fish FCR and survival comprises administering to the fish a non-peptidic TK antagonist.

Any know non-peptidic fish TK antagonist may be used according to the method of the present invention.

According to some specific embodiments, the non-peptidic NKB antagonist selected from the group consisting of: (S)-(2)-N-(a-ethylbenzyl)-3-methyl-2-phenylquinoline-4-carboxamide (also denoted SB-222200); (S)-(1)-N-{{3-[1-benzoyl-3-(3,4-dichlorophenyl)piperidin-3-yl]prop-1-yl}-4-phenylpiperidin-4-yl}-N-methylacetamide (also denoted Osanetant and SR-142,801), and (S)-(2)-N-(a-ethylbenzyl)-3-hydroxy-2-phenylquinoline-4-carboxamide (also denoted talnetant and SB 223412). Each possibility represents a separate embodiment of the present invention.

Any administering route or mode can be used for providing the TK antagonist or composition comprising it, to fish according to the methods of the present invention. This include parenteral administration and enteral administration. According to some embodiments, parenteral administration includes but is not limited to any type of injection. According to some embodiments, enteral administration includes but is not limited to oral administration, including administration as additive to the food, administration by immersion (e.g. through the skin or the gils and including administration as additive to the drinking water), and intragastric administration via gavage.

According to some embodiments the method comprises administering the composition to fish as part of regular feed or water consumption.

According to some specific embodiments, the method comprises administering the TK antagonist as coating of feed pellets.

According to some embodiments, the feed pellets are coated with a final TK antagonist dose of 1-50 mg per one kg of feed.

According to other specific embodiments, the feed pellets are coated with a final TK antagonist dose of 2-40 mg per one kg of feed.

According to other specific embodiments, the feed pellets are coated with a final TK antagonist dose of 5-30 mg per one kg of feed.

According to other specific embodiments, the feed pellets are coated with a final TK antagonist dose of 5-10 mg per one kg of feed.

According to some embodiments, the method comprises administering the composition to fish in a volume of water to be taken up by the gills or to be absorbed by the skin.

According to other embodiments, administration of the composition is orally.

The present invention provides, according to another aspect, a composition comprising at least one TK antagonist, for use in improving fish FCR or at least one fish health parameter selected from the group consisting of: survival, skin condition and resistance to pathogens.

According to some embodiments, the composition comprising a TK antagonist, for use in improving fish FCR and survival, is selected from a pharmaceutical composition and a feed composition.

According to some embodiments the composition comprises a peptidomimetic according to Formula I wherein X₁ comprises at least one aromatic amino acid residue in L or D configuration.

According to some embodiments the composition comprises a peptidomimetic according to Formula I wherein X₁ comprised a D-Trp residue.

According to some embodiments the composition comprises a peptidomimetic according to Formula I wherein X₁ comprises at least one negatively charged (acidic) amino acid residue.

According to some embodiments the composition comprises a peptidomimetic according to Formula I wherein the C-terminus is amidated.

According to some embodiments the composition comprises a peptidomimetic according to Formula I wherein X₁ consists of 2 or 3 amino acids and a capped N-terminus. Each possibility represents a separate embodiment of the present invention.

According to some embodiments the composition comprises a peptidomimetic according to Formula I wherein X₁ consists of 2 or 3 amino acid residues comprising an aromatic residue, a negatively charged (acidic) residue, an amidated C-terminus and an N-terminus capping moiety. Each possibility represents a separate embodiment of the present invention.

According to some embodiments the composition comprises a peptidomimetic according to Formula I wherein X₁ comprises a residue selected from an aliphatic amino acid residue and a polar, uncharged residue. Each possibility represents a separate embodiment of the present invention.

According to some embodiments the composition comprises a peptidomimetic according to Formula I wherein the aliphatic residue is selected from the group consisting of: Ala, Ile, and Leu. Each possibility represents a separate embodiment of the present invention.

According to some embodiments the composition comprises a peptidomimetic according to Formula I wherein the polar, uncharged residue is selected from Ser and Thr. Each possibility represents a separate embodiment of the present invention.

According to some embodiments the composition comprises a peptidomimetic according to Formula I wherein X₁ comprises an aromatic residue selected from the group consisting of Phe, DPhe, Trp and DTrp; a negatively charged (acidic) residue selected from Glu and Asp; and a succinyl (Succ)N-terminus capping moiety. Each possibility represents a separate embodiment of the present invention.

According to some embodiments the composition comprises a peptidomimetic according to Formula I wherein X₁ comprises an aromatic residue selected from Phe, and DTrp; an Asp residue; a succinyl (Succ)N-terminus capping moiety, and optionally a residue selected from Ile and Ser. Each possibility represents a separate embodiment of the present invention.

According to some embodiments, the composition comprises a peptidomimetic according to formula II:

X₁—NMeVal-βAla-Leu-Met-NH₂ (Formula II);

wherein, X₁ is selected from the group consisting of: Succ-Asp-Phe; Succ-Asp-DPhe; Succ-Asp-Trp; Succ-Asp-DTrp; Succ-Asp-Ile-Phe; Succ-Asp-Ile-DPhe; Succ-Asp-Ile-Trp; Succ-Asp-Ile-DTrp; Succ-Asp-Ser-Phe; Succ-Asp-Ser-DPhe; Succ-Asp-Ser-Trp; Succ-Asp-Ser-DTrp, Succ-Glu-Phe; Succ-Glu-DPhe; Succ-Glu-Trp; Succ-Glu-DTrp; Succ-Glu-Ile-Phe; Succ-Glu-Ile-DPhe; Succ-Glu-Ile-Trp; Succ-Glu-Ile-DTrp; Succ-Glu-Ser-Phe; Succ-Glu-Ser-DPhe; Succ-Glu-Ser-Trp; and Succ-Glu-Ser-DTrp. Each possibility represents a separate embodiment of the present invention.

According to some embodiments the composition comprises a peptidomimetic consisting of 5-10 amino acid residues comprising the sequence NMeVal-βAla-Leu-Met (SEQ ID NO. 7).

According to some embodiment the composition comprises a peptidomimetic comprising a sequence of SEQ ID NO. 7, at least one aromatic amino acid residue and at least one negatively charged amino acid residue.

According to some embodiments, the composition comprises a peptidomimetic comprising a sequence of SEQ ID NO. 7, at least one aromatic amino acid residue, at least one negatively charged amino acid residue and at least one residue selected from an aliphatic amino acid residue and a polar, uncharged residue. Each possibility represents a separate embodiment of the present invention.

According to some embodiments, the composition comprises a peptidomimetic comprising a capped N-terminus.

According to some embodiments the composition comprises a peptidomimetic comprising an amidated C-terminus.

According to some embodiments the composition comprises a peptidomimetic consisting of 5, 6, 7, 8, 9 or 10 amino acid residues and an optional N-terminal capping group. Each possibility represents a separate embodiment of the present invention.

According to some embodiments, the composition comprises a peptidomimetic consisting of 5-10 amino acid residues, an amidated C-terminus and an N-terminal capping moiety. Each possibility represents a separate embodiment of the present invention.

According to some embodiments, the composition comprises a peptidomimetic consisting of 6-7 amino acid residues comprising the sequence of SEQ ID NO. 7, and a sequence selected from the group consisting of: Succ-Asp-Phe; Succ-Asp-DPhe; Succ-Asp-Trp; Succ-Asp-DTrp; Succ-Asp-Ile-Phe; Succ-Asp-Ile-DPhe; Succ-Asp-Ile-Trp; Succ-Asp-Ile-DTrp; Succ-Asp-Ser-Phe; Succ-Asp-Ser-DPhe; Succ-Asp-Ser-Trp; Succ-Asp-Ser-DTrp, Succ-Glu-Phe; Succ-Glu-DPhe; Succ-Glu-Trp; Succ-Glu-DTrp; Succ-Glu-Ile-Phe; Succ-Glu-Ile-DPhe; Succ-Glu-Ile-Trp; Succ-Glu-Ile-DTrp; Succ-Glu-Ser-Phe; Succ-Glu-Ser-DPhe; Succ-Glu-Ser-Trp; and Succ-Glu-Ser-DTrp.

According to some embodiments the at least one N-terminal capping moiety is selected from the group consisting of: succinyl, oxalyl, malonyl, glutaryl, adipoyl, pimaloyl, suberoyl, acetyl, and other dicarboxylic acid residues. Each possibility represents a separate embodiment of the present invention.

According to some specific embodiments the composition for use in improving fish FCR and/or survival comprises a peptidomimetic selected from the group consisting of:

(SEQ ID NO. 1, AN1)
Succ-Asp-Ile-Phe-N(Me)Val-βAla-Leu-Met-NH₂;
(SEQ ID NO. 2, AN2)
Succ-Asp-Phe-N(Me)Val-βAla-Leu-Met-NH₂;
(SEQ ID NO. 3, AN3)
Succ-Asp-Ser-Phe-N(Me)Val- βAla-Leu-Met-NH₂;
(SEQ ID NO. 4, AN4)
Succ-Asp-Ile-D-Trp-N(Me)Val- βAla-Leu-Met-NH₂;
(SEQ ID NO. 5, AN5)
Succ-Asp-D-Trp-N(Me)Val- βAla-Leu-Met-NH₂;
and
(SEQ ID NO. 6, AN6)
Succ-Asp-Ser- D-Trp -N(Me)Val-βAla-Leu-Met-NH₂;

wherein Succ denotes a succinyl.

Each possibility represents a separate embodiment of the present invention.

According to some embodiments, the composition for use in improving fish FCR and/or survival, comprises a non-peptidic TK antagonist.

According to some specific embodiments, the composition for use in improving fish FCR and/or survival comprises a non-peptidic NKB antagonist selected from the group consisting of: (S)-(2)-N-(a-ethylbenzyl)-3-methyl-2-phenylquinoline-4-carboxamide (also denoted SB-222200); (S)-(1)-N-{{3-[1-benzoyl-3-(3,4-dichlorophenyl)piperidin-3-yl]prop-1-yl}-4-phenylpiperidin-4-yl}-N-methylacetamide (also denoted Osanetant and SR-142,801), and (S)-(2)-N-(a-ethylbenzyl)-3-hydroxy-2-phenylquinoline-4-carboxamide (also denoted talnetant and SB 223412). Each possibility represents a separate embodiment of the present invention.

A pharmaceutical composition according to the invention, for use in improving fish FCR and/or survival, comprises a TK antagonist and an optional acceptable carrier, diluent, salt or excipient.

A feed composition according to the present invention, for use in improving fish FCR and/or survival, comprises a TK antagonist and an optional food additive. Any food additive known in the art may be used in a food composition according to the invention. This includes but is not limited to color additives, taste additives etc. Nutrients, including but not limited to proteins, carbohydrates, fats minerals, vitamins etc., may be also included in the food compositions of the present invention. A food composition according to the invention may comprise nutrients and/or food additives in addition to at least one TK antagonist.

According to some embodiments, the feed composition comprises feed pellets coated with at least one TK antagonist.

According to some embodiments, the feed composition is extruded feed comprising at least one TK antagonist.

According to some specific embodiments, the feed pellets are coated with a final TK antagonist dose of 1-50 mg per one kilogram of feed.

According to other specific embodiments, the feed pellets are coated with a final TK antagonist dose of 5-30 mg per one kilogram of feed.

According to other specific embodiments, the feed pellets are coated with a final TK antagonist dose of 5-10 mg per one kilogram of feed.

According to some embodiments, the peptide or peptides are added to the feed during its preparation in an extruder.

According to some specific embodiments, a feed composition is provided, comprising the TK antagonist of the sequence Succinyl-Asp-Ser-D-Trp—N(Me)Val-βAla-Leu-Met-NH₂ (SEQ ID NO. 6, AN6), for use in improving fish FCR and survival.

A composition comprising a TK antagonist for improving fish FCR according to the invention, may be administered to fish by any manner or route known in the art including parenteral administration and enteral administration. According to some embodiments, parenteral administration includes but is not limited to any type of injection. According to some embodiments, enteral administration includes but is not limited to oral administration, including administration as additive to the food, administration by immersion, including administration as additive to the drinking water, absorption by skin, and intragastric administration via gavage.

According to some the composition is administered to fish orally as part of regular feed or water consumption.

According to some embodiments, the composition is administered to fish in a volume of water to be taken up by the gills or to be absorbed by the skin.

Fish according to the invention include any type of fish from any class, subclass, order, family or genus including farmed fish, edible fish and ornamental fish. According to some non-limitative embodiments, the fish is selected from the group consisting of: tilapia, carp, salmon, bass, catfish and mullet.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 represents the effect of supplementing AN6 peptide to fish feeds at different concentrations and ages on daily grow rate, as determined by two different experiments, #1 and #2, detailed in Examples 2 and 3.

FIG. 2A represents the effect of supplementing AN6 peptide to fish feeds at different concentrations and ages on weight gain, in experiment #1.

FIG. 2B represents the effect of supplementing AN6 peptide to fish feeds at different concentrations and ages on weight gain, in experiment #2.

FIG. 3A represents the effect of supplementing AN6 peptide to fish feeds at different concentrations and ages on total biomass in experiment #1.

FIG. 3B represents the effect of supplementing AN6 peptide to fish feeds at different concentrations and ages on total biomass in experiment #2.

FIG. 4 shows the effect of supplementing AN6 peptide to fish feeds in amounts of 5 and 10 mg/Kg for 90 days (60-150 dph) on growth of 8-month-old male fish (244 fish in control group, 253 fish treated with 5 mg/mg, 292 fish treated with 10 mg/kg) and female fish (215 fish in control group, 199 fish treated with 5 mg/mg, 165 fish treated with 10 mg/kg).

FIG. 5A represents the effect of supplementing AN6 peptide to fish feeds in amounts of 10 mg/Kg for 90 days (10-106 dph) and 30 mg/Kg (207-256 dph) on fish feed conversion rate (experiment #1). Values indicate percentage change relative to control of FCR in response to TK antagonist treatment.

FIG. 5B shows the effect of supplementing AN6 peptide to fish feeds in ratio of 5 and 10 mg/Kg for 90 days (60-150 dph) on fish FCR (experiment #2). Values indicate percentage change relative to control of FCR in response to TK antagonist treatment.

FIG. 6 represents the effect of supplementing AN6 peptide to fish feeds in ratio of 5 and 10 mg/Kg on intestine weight (experiment #2).

FIG. 7 represents the effect of supplementing AN6 peptide to fish feeds on fish survival (experiment #2).

FIG. 8 represents the effect of supplementing AN6 peptide to fish feeds at different doses for various lengths of time on average fish weight per cage. Values indicate percentage change of average fish weight per cage for the indicated AN6 treatment schedules relative to control (experiment #3). The different parameters of treatment schedules 1 to 8 are detailed in Example 5.

FIG. 9A represents the effect of supplementing AN6 peptide to fish feeds at different doses for various lengths of time on average fish growth rate per day. Values indicate percentage change of average fish growth rate per day for the indicated AN6 treatment schedules relative to control (experiment #3). The different parameters of treatment schedules 1 to 8 are detailed in Example 5.

FIG. 9B represents the effect of supplementing AN6 peptide to fish feeds at different doses for various length of time on average fish growth rate over time. Values indicate percentage change of average fish growth rate over time per indicated AN6 treatment schedule relative to control (experiment #3). The different parameters of treatment schedules 1 to 8 are detailed in Example 5.

FIG. 10 represents the effect of supplementing AN6 peptide to fish feeds in amounts of 10 mg/Kg feed for 90 days (10-106 dph) and 30 mg/Kg feed (207-256 dph) (Groups A and B, Phase 1 and 2) on average FCR over time (experiment #1).

FIG. 11 represents the effect of supplementing AN6 peptide to fish feeds in amounts of 5 or 10 mg/Kg feed for 90 days (60-150 DPH) (Groups C, D, and E) on average FCR over time (experiment #2).

FIG. 12 represents the effect of supplementing AN6 peptide to fish feeds at different doses for various lengths of time on average FCR over time. Values indicate percentage change of average fish weight per cage for the indicated AN6 treatment schedules relative to control (experiment #3). The different parameters of treatment schedules 1 to 8 are detailed in Example 5.

FIG. 13 represents the effect of different concentrations of AN6 on the binding of Senktide to NKB receptors in COS7 cells. Naïve COS7 cells were transfected with DNA of Tilapia Tac3Ra (t1NKB receptor a, cloned in pcDNA3.1). NKB receptors were activated by Senktide, a potent synthetic and specific NKB activator. Receptor binding was determined by dynamic reads of fluorescent emission from a Ca++ indicator reporter system (Fluo4AM, Molecular Probes). Relative fluorescence was calculated for the indicated concentrations of AN6. AN6 inhibition affinity (IC50), the concentration required to reduce by half the average florescence emission induced by agonist in constant concentration, is indicated.

FIG. 14 represents the effect of different concentrations of AN3 on the binding of Senktide to NKB receptors in COS7 cells. Naïve COS7 cells were transfected with DNA of Tilapia Tac3Ra (t1NKB receptor a, cloned in pcDNA3.1).

FIG. 15 represents the effect of different concentrations of AN5 on the binding of Senktide to NKB receptors in COS7 cells. Naïve COS7 cells were transfected with DNA of Tilapia Tac3Ra (t1NKB receptor a, cloned in pcDNA3.1).

FIG. 16 represents the effect of different concentrations of AN4 on the binding of Senktide to NKB receptors in COS7 cells. Naïve COS7 cells were transfected with DNA of Tilapia Tac3Ra (t1NKB receptor a, cloned in pcDNA3.1).

FIG. 17 represents the effect of different concentrations of AN3 on the activity of NKB receptors in COS7 cells. Naïve COS7 cells were co-transfected with DNA of Salmon Tac3R and Luciferase reporter under the regulation of SRE promoter. Nuclear SRE promoter activation is part of the PKA signaling pathway that is typical for NKB receptor activation. Salmon NKB receptor activity is determined by reading of Luciferase fluorescence emission in plate reader 6 hours after activation. NKB receptors were activated by Senktide, a potent synthetic and specific NKB activator, simultaneously with AN3 treatment. Relative fluorescence was calculated for the indicated concentrations of AN3. AN3 inhibition affinity (IC50), the concentration required to reduce by half the average florescence emission induced by agonist in constant concentration, is indicated.

FIG. 18 represents the effect of different concentrations of AN2 on the activity of NKB receptors in COS7 cells. Naïve COS7 cells were co-transfected with DNA of Salmon Tac3R and Luciferase reporter under the regulation of SRE promoter.

### DETAILED DESCRIPTION OF THE INVENTION

The present invention provides methods for improving fish feed conversion rate and survival and for increasing fish growth profitability. The invention is based on inhibition of tachykinin receptor activity in the GI track and in the nervous system of fish.

Without wishing to be bound to any mechanism of action, it is proposed that the TK antagonists act via at least one mechanism selected from the group consisting of: reducing gut motility, increasing feed retention time in the gut, modulating secretion of electrolytes or digestion fluids from gut epithelial cells, influencing proliferation of gut cells, mitigating the adverse effects of inflammation on gut function and affecting microbial population in the gut.

It is also proposed, that TK antagonists protect fish from stress-induces processes, thereby improving overall health and survival.

Without wishing to be bound to any mechanism of action, it is also proposed that treatment of fish with TK antagonists may also result in increase in intestinal size of the fish.

It should be understood that treated fish do not necessarily gain extra weight following treatment with a TK antagonist, but rather a lower amount of feed is required by a TK antagonist-treated fish, to gain the same weight as an untreated fish from the same species and age. Effect on FCR is not related to the fish sexual maturation state, both adult/sexually mature fish and young fish are affected by and eligible for such TK antagonist treatment. Improvement of fish FCR, overall health or survival according to the present invention does not involve alteration of any piscine reproduction system parameter.

A TK antagonist according to the invention is a compound capable of binding to a piscine tachykinin receptor and inhibiting its activity. TK antagonists of the present invention may inhibit at least one NK receptor type present in fish, including but not limited to NK1R, NK2R and NK3R, either by orthosteric or allosteric binding.

A TK antagonist according to the present invention may be a peptide-based molecule, such as peptide, polypeptide or peptidomimetic, or a non-peptidic molecule.

A TK antagonist according to the present invention may be synthesized by any method known in the art including synthetic and recombinant production methods (when a peptide containing only natural amino acids is produced). Peptide-based TK antagonists may be produced, according to some embodiments, using solid or liquid phase synthesis. The design and synthesis of several TK antagonist peptidomimetics is described for example in WO 2016/009439.

Feed conversion rate or ratio (FCR) is weight to weight ratio between feed consumed by fish in a specific period and fish body mass gained at this period therefore indicates the efficiency of a feed or a feeding strategy. Improvement of FCR means decrease in its value, namely a low FCR indicate that it takes less feed to produce one kilogram of fish than it would if the FCR were higher. The FCR allows for an estimate of the feed that will be required in a growing cycle and therefore assists in predicting the profitability of the production. Decreasing FCR values generally leads to increased profitability since feed is the highest expense in fish farming. FCR is influenced by several parameters, such as water quality, fish health, method of feeding, temperature etc., and is generally lower in small fish than in the same species of fish that is larger. Low FCR also result in less waste polluting the pond water. With better water quality, the carrying capacity of the pond is increased (https://pdf.usaid.gov/pdf_docs/PA00K8MQ.pdf).

The physiology underlining FCR improvement disclosed in the methods of the present invention can be broken into several successive steps: Feed Intake→Digestion→Absorption→Conversion. While digestion and absorption relate to gut physiology, conversion occurs mainly in the soma and is determined by the availability of metabolites and energy in growth processes on the expanse of other energy consuming processes (metabolism).

In the relationship between FCR and growth, two main dogmas are possible: a. Improved FCR value which is a manifestation of similar growth performance with less feed consumed. b. Improved FCR that occurs when fish have better growth with similar or higher feed intake. Namely, improved FCR does not necessarily result in an increased growth rate, and vice versa—increased growth rate does not necessarily indicate an improved FCR. According to the first dogma, feed is digested and absorbed in higher efficiency, but growth potential is not altered. In the second dogma, a shift in metabolism towards growth may also be involved. Consistent with these scenarios, the present invention demonstrates different FCR-growth relationships: improved FCR and growth performance, and improved FCR which did not coincide with improved growth but rather with decrease in feed intake. These results suggest that TK antagonists act on both levels of gut physiology and metabolism, but that the effect on metabolism is evidential only at certain regimes.

The results of the present invention also suggest that treatment with TK antagonists improves fish immune response, mainly in the GI tract, and reduced stress levels resulting in better survival of the fish.

There are 3 TK receptors: tac1 (high affinity to SP), tac2 (high affinity to NKA) and tac3 (high affinity to NKB). Results presented herein demonstrate that the expression of all TK receptors (tac1, tac2, tac3) are altered in the different parts of the gut and in the skin following AN6 treatment. Without wishing to be bound to any mechanism of action, it is proposed that AN6 effect on fish physiology is mediated by more than one TK receptor and optionally, 2 or all 3 receptors are involved, with expected differences between tissues. The following observations suggest that AN6 effect is mediated primarily by local receptors in the gut: TK receptors mRNA expression (tac1, tac2, tac3) is altered in the different parts of the gut following AN6 treatment, suggesting local effect on gut physiology; AN6 is cleared from blood very quickly (˜4.5 min); there is no effect on gonadal state when administering 10-30 ppm of AN6; effect on growth and FCR observed even before sexual maturation (before 90 dph) and no peptide residual is detected in muscle and blood after treatment.

The contribution of tachykinins to homeostasis and diseases of the skin is well established (Scholzen et al., Exp Dermatol, 7(2-3):81-961998). The involvement of TK in fish skin pigmentation is addressed in Huali et al. 2018 (The FASEB Journal 32(6):3193-3214). Skin health has great importance for the aquaculture of edible fish (e.g., salmon and trout) as well as for ornamental fish farming. According to some embodiments, the methods and compositions of the present invention are used for improving skin health.

Fish age is determined in the present invention as days post hatching (dph). For example, in tilapia, early juvenile, characterized by self-feeding and specific coloration is about 7-25 dph; late juvenile characterized by gradual disappearance of specific coloration and by maturation of the gametes, is between 25-95 dph; while adult tilapia fish are usually about 96 dph. In other fish types growth stages are at different dph. Effective treatment age of fish depends on the fish type. Tilapia are treated at age 0 to 4 month and marketed at 8 to 10 months while salmon are marketed at age of 2-3 years and treatment can occur anywhere in until that age.

The experiments of the present invention suggest that effects on reproduction and growth are not linked and that the compounds of the present invention are safe when administered to fish and do not influence the reproductive system. Histological data on fish gonads indicate that gonads were normally developed in the treated fish and that TK receptor (Tac) expression levels are not altered due to treatment.

According to some embodiments, gender specific effects may be different in other type of fish.

According to some specific embodiments, the method of the present invention comprises administering as an active ingredient a compound selected from the group consisting of:

(SEQ ID NO. 1, AN1)
Succ-Asp-Ile-Phe-N(Me)Val-βAla-Leu-Met-NH₂;
(SEQ ID NO. 2, AN2)
Succ-Asp-Phe-N(Me)Val-βAla-Leu-Met-NH₂;
(SEQ ID NO. 3, AN3)
Succ-Asp-Ser-Phe-N(Me)Val- βAla-Leu-Met-NH₂;
(SEQ ID NO. 4, AN4)
Succ-Asp-Ile-D-Trp-N(Me)Val- βAla-Leu-Met-NH₂;
(SEQ ID NO. 5, AN5)
Succ-Asp-D-Trp-N(Me)Val- βAla-Leu-Met-NH₂;
and
(SEQ ID NO. 6, AN6)
Succ-Asp-Ser- D-Trp -N(Me)Val-βAla-Leu-Met-NH₂;

The peptides of the present invention are preferably synthesized using conventional synthesis techniques known in the art, e.g., by chemical synthesis techniques including peptidomimetic methodologies. These methods include exclusive solid phase synthesis, partial solid phase synthesis methods, fragment condensation, classical solution synthesis. Solid phase peptide synthesis procedures are well known in the art. A skilled artesian may synthesize any of the peptides of the present invention by using an automated peptide synthesizer using standard chemistry such as, for example, t-Boc or Fmoc chemistry. Synthetic peptides can be purified by preparative high-performance liquid chromatography, and the composition of which can be confirmed via amino acid sequencing. Conjugation of peptidic and permeability moieties may be performed using any methods known in the art, either by solid phase or solution phase chemistry. Some of the preferred compounds of the present invention may conveniently be prepared using solution phase synthesis methods. Other methods known in the art to prepare compounds like those of the present invention can be used and are comprised in the scope of the present invention.

N-terminal capping or modification according to the present invention denotes alteration of the peptide's sequence by covalently attaching a chemical moiety to the terminal amine resulting in modified charge, activity and/or stability to cleavage by amino peptidases.

Non-limitative examples of a permeability-enhancing moiety include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to some embodiments, the hydrophobic moiety is a lipid moiety or an amino acid moiety.

A permeability-enhancing moiety may be connected to any position in the peptide moiety, directly or through a spacer. According to specific embodiments, the cell-permeability moiety is connected to the amino terminus of the peptide moiety. The optional connective spacer may be of varied lengths and conformations comprising any suitable chemistry including but not limited to amine, amide, carbamate, thioether, oxyether, sulfonamide bond and the like. Non-limiting examples for such spacers include amino acids, sulfone amide derivatives, amino thiol derivatives and amino alcohol derivatives.

The term “peptide” or “peptide-based” as used herein is meant to encompass natural (genetically encoded), non-natural and/or chemically modified amino acid residues, each residue being characterized by having an amino and a carboxy terminus, connected one to the other by peptide or non-peptide bonds. The amino acid residues are represented throughout the specification and claims by either one or three-letter codes, as is commonly known in the art. The peptides and peptidomimetics of the present invention are preferably utilized in a linear form, although it will be appreciated that in cases where cyclization does not severely interfere with peptide characteristics, cyclic forms of the peptide can also be utilized.

The amino acids used in this invention are those which are available commercially or are available by routine synthetic methods. Certain residues may require special methods for incorporation into the peptide, and sequential, divergent or convergent synthetic approaches to the peptide sequence are useful in this invention. Natural coded amino acids and their derivatives are represented by three-letter codes according to IUPAC conventions. When there is no indication, either the L or D isomers may be used. When a “D”—precedes the amino acid, a D isomer is used.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, affinity to the target protein, metabolic stability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following is an example of classification of the amino acids into six groups, each contains amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), methionine (M), Valine (V); and

6) Phenylalanine (F), Tyrosine (Y), Tryptophan (W).

Other classifications into somehow different groups (for example, aliphatic, polar, non-polar, hydrophilic, hydrophopic etc.) are also known in the art and can be used for conservative amino acid substitutions according to the present invention.

Also included within the scope of the invention are salts of the peptides, analogs, and chemical derivatives of the peptides of the invention.

As used herein the term “salts” refers to both salts of carboxyl groups and to acid addition salts of amino or guanido groups of the peptide molecule. Salts of carboxyl groups may be formed by means known in the art and include inorganic salts, for example sodium, calcium, ammonium, ferric or zinc salts, and the like, and salts with organic bases such as salts formed for example with amines such as triethanolamine, piperidine, procaine, and the like. Acid addition salts include, for example, salts with mineral acids such as, for example, acetic acid or oxalic acid. Salts describe here also ionic components added to the peptide solution to enhance hydrogel formation and/or mineralization of calcium minerals.

A “chemical derivative” as used herein refers to peptides containing one or more chemical moieties not normally a part of the peptide molecule such as esters and amides of free carboxy groups, acyl and alkyl derivatives of free amino groups, phospho esters and ethers of free hydroxy groups. Such modifications may be introduced into the molecule by reacting targeted amino acid residues of the peptide with an organic derivatizing agent that is capable of reacting with selected side chains or terminal residues. Preferred chemical derivatives include peptides that have been phosphorylated, C-termini amidated or N-termini acetylated.

“Functional derivatives” of the peptides of the invention as used herein covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it and do not adversely affect the antigenic properties thereof. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “peptide analog” indicates molecule which has the amino acid sequence according to the invention except for one or more amino acid changes or one or more modification/replacement of an amide bond. Peptide analogs include amino acid substitutions and/or additions with natural or non-natural amino acid residues, and chemical modifications which do not occur in nature. Peptide analogs include peptide mimetics. A peptide mimetic or “peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “analogs” may be computer assisted. Additional peptide analogs according to the present invention comprise a specific peptide or peptide analog sequence in a reversed order, namely, the amino acids are coupled in the peptide sequence in a reverse order to the amino acids order which appears in the native protein or in a specific peptide or analog identified as active. Whether completely or partially non-peptide, peptidomimetics according to this invention provide a spatial arrangement of chemical moieties that closely resembles the three-dimensional arrangement of groups in the peptide on which the peptidomimetic is based. As a result of this similar active-site structure, the peptidomimetic has effects on biological systems, which are similar to the biological activity of the peptide.

A modified amino acid residue is an amino acid residue in which any group or bond was modified by deletion, addition, or replacement with a different group or bond, as long as the functionality of the amino acid residue is preserved or if functionality changed (for example replacement of tyrosine with substituted phenylalanine) as long as the modification did not impair the activity of the peptide containing the modified residue.

“A peptide conjugate” according to the present invention, denotes a molecule comprising a TK antagonistic peptide to which another moiety, either peptidic or non peptidic, is covalently bound, directly or via a linker. According to some embodiments, a permeability-enhancing moiety, e.g. a fatty acid residue is bound to the peptidic TK antagonist.

The term “linker” denotes a chemical moiety, a direct chemical bond of any type, or a spacer whose purpose is to link, covalently, a cell-permeability moiety and a peptide or peptidomimetic. The spacer may be used to allow distance between the permeability-enhancing moiety and the peptide.

“Permeability” refers to the ability of an agent or substance to penetrate, pervade, or diffuse through a barrier, membrane, or a skin layer. A “cell permeability” or a “cell-penetration” moiety refers to any molecule known in the art which is able to facilitate or enhance penetration of molecules through membranes. Non-limitative examples include: hydrophobic moieties such as lipids, fatty acids, steroids and bulky aromatic or aliphatic compounds; moieties which may have cell-membrane receptors or carriers, such as steroids, vitamins and sugars, natural and non-natural amino acids, transporter peptides, nanoparticles and liposomes.

The term “physiologically acceptable carrier” or “diluent” or “excipient” refers to an aqueous or non-aqueous fluid that is well suited for pharmaceutical preparations. Furthermore, the term “a pharmaceutically acceptable carrier or excipient” refers to at least one carrier or excipient and includes mixtures of carriers and or excipients. The term “therapeutic” refers to any pharmaceutical, drug or prophylactic agent which may be used in the treatment (including the prevention, diagnosis, alleviation, or cure) of a malady, affliction, disease or injury in a patient.

### Pharmacology

Apart from other considerations, the fact that the novel active ingredients of the invention are peptides, peptide analogs or peptidomimetics, dictates that the formulation be suitable for delivery of these types of compounds. Although in general peptides are less suitable for oral administration due to susceptibility to digestion by gastric acids or intestinal enzymes novel methods are being used, in order to design and provide metabolically stable and oral bioavailable peptidomimetic analogs.

The pharmaceutical composition of this invention may be administered by any suitable means, such as orally, topically, intranasally, subcutaneously, intramuscularly, intravenously, intra-arterially, intraarticulary, intralesionally, by inhalation or parenterally, and are specifically formulated for the administration route. The compositions are formulated according to the administration route.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p.1).

The doses for administration of such pharmaceutical compositions range according to some embodiments of the present invention from about 0.1 mg/kg to about 50 mg/kg body weight.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

In certain embodiments, peptide delivery can be enhanced by the use of protective excipients. This is typically accomplished either by complexing the peptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy, 1998, Biotechnol. Prog. 14, 108; Johnson et al., 1996, Nature Med. 2, 795; Herbert et al., 1998, Pharmaceut. Res. 15, 357) a dry powder composed of biodegradable polymeric microspheres containing the protein in a polymer matrix that can be compounded as a dry formulation with or without other agents.

In certain embodiments, dosage forms of the compositions of the present invention include, but are not limited to, biodegradable injectable depot systems such as, PLGA based injectable depot systems; non-PLGA based injectable depot systems, and injectable biodegradable gels or dispersions. Each possibility represents a separate embodiment of the invention. The term “biodegradable” as used herein refers to a component which erodes or degrades at its surfaces over time due, at least in part, to contact with substances found in the surrounding tissue fluids, or by cellular action. In particular, the biodegradable component is a polymer such as, but not limited to, lactic acid-based polymers such as polylactides e.g. poly (D,L-lactide) i.e. PLA; glycolic acid-based polymers such as polyglycolides (PGA) e.g. Lactel® from Durect; poly (D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as Poly(e-caprolactone) i.e. PCL (Lactel® from Durect); polyanhydrides; poly(sebacic acid) SA; poly(ricenolic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy} propane), CPP; poly(p-{carboxyphenoxy} hexane) s CPH; polyamines, polyurethanes, polyesteramides, polyorthoesters {CHDM: cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkylene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly (N-isopropylacrylamide) (PNIPAAm), poly (ethylene oxide)-poly (propylene oxide)-poly (ethylene oxide) tri-block copolymers (PEO—PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate) and the like, or mixtures thereof.

In some embodiments, the compositions of the present invention comprise a biodegradable polymer selected from, but not limited to, PLGA, PLA, PGA, polycaprolactone, polyhydroxybutyrate, polyorthoesters, polyalkaneanhydrides, gelatin, collagen, oxidized cellulose, polyphosphazene and the like. Each possibility represents a separate embodiment.

### Fish Feed Compositions

The feed compositions of the present invention may be part of, or mixed with conventional of specific fish feed. Fish feed is normally specific for the type of fish to be nourished and includes proteins, oils, vitamins and other additives.

According to some non-limiting examples, the compositions of the present invention are pelletized, solid compositions containing, about 15 to 60 percent protein or protein hydrolysate, about 2 to 15 percent fat, or about 3 to 10 percent crude fiber together with adjuvants, such as minerals, vitamins, and/or trace elements. According to certain embodiments, the feed compositions contain 25-55 percent protein and 15-25 percent lipid (fat). According to some embodiments, the feed compositions further comprise salts, betaine, amino acids, nucleotides and other supplements to improve the osmotic adaptation of young fish to seawater and for better survival. According to further embodiments, the feed composition supplied to the fish is changed during the period of fish growth, for example, the protein content is decreased and the lipid content is increased during the seawater grow-out phase.

Fish feed composition comprising the antagonists of the present invention are prepared using methods known in the art. For example, pellets, typically 0.5-20 millimeters in diameter, are made from the composition and dried prior to storage and use. The coherence of the pellets may be improved by dissolving a small amount of gelatin in the water. If water-soluble whey powder provides much of the protein content, the pellet surfaces are preferably coated with a little oil or fat to prevent premature disintegration of the pellets upon contact with water. Gelatin-bearing compositions may be foamed in a conventional manner to produce cellular pellets whose overall density is similar to that of water. Such pellets float in water and remain accessible to the fish for a relatively long period. Pellets that sink to the bottom are lost to many fish. The antagonists of the invention may also be mixed with commercial fish feed of conventional composition by uniformly distributing the antagonists in the conventional composition and thereafter making pellets from the mixture obtained. According to some embodiments, feed pellets are coated with the compounds of the invention and used for feeding the fish.

The feed compositions of the present invention may be prepared by extrusion, a process by which a feed or food material is pushed, using a piston or screw, through an orifice or a die of a given shape and size. Extruders are available in several designs depending on their application, but are generally classified based on the number of screws. The two general types of extruders are single-screw and twin-screw configurations. Single screw extruders operate based on the pressure requirement of the die, slip at the barrel wall and the extent to which the screw is filled, whereas twin screw extruders operate based on the direction the two screws rotate and the extent of intermeshing between the two screws. There are two main extrusion process: dry process, wherein the heat from friction of materials is used to force the materials to pass through the die holes; and wet process, similar to the dry process but with addition of water and vapor up to 20-30% moisture level. Any extruder and extruding method may be used for preparing feed compositions according to the present invention.

Although the present invention has been described with respect to various specific embodiments thereof in order to illustrate it, such specifically disclosed embodiments should not be considered limiting. Many other specific embodiments will occur to those skilled in the art based upon applicants' disclosure herein, and applicants propose to be bound only by the spirit and scope of their invention as defined in the appended claims.

### EXAMPLES

The following examples demonstrate the production and in-vivo activity of TK antagonists in improving fish FCR and health and reducing overall mortality.

Three separate experiments (#1, #2 and #3) on fish are described in the following examples. Experiment #1 studied the effects of supplementing fish feed with two different doses of AN6 peptide in two phases, from 10 dph for 90 days and from 207 dph for 50 days. Experiment #2 examined the effects of supplementing fish feed with either 5 or 10 mg AN6/kg different doses of AN6 from 60 dph for 90 days. Experiment #3 examined the effects of supplementing fish feed starting from 10 or 90 dph with either 5, 10, or 30 mg AN6/kg for various lengths of time. Results were analyzed for a number of growth-related parameters.

### Example 1—Feed Coating

MeM (powder, manufactured by Bern Aqua), Nanolis P1, P2 (pellet sizes of 0.8-1 mm, 1-1.5 mm, respectively, manufactured by Ocialis) and Tilaphi 1, 2 & 3 (pellet sizes of 1-1.5 mm, 2 mm, 3-5.5 mm, respectively, manufactured by Ocialis) were used as basic regular feed. The basic feed was coated according to the following general procedure: 9 ml or 3 ml of Tris-HCl solution (50 mM, pH=8.8) were diluted to 60 ml with distilled H₂O. For AN6-containing feed preparation, desired amounts of AN6 peptide (a TK antagonist having the sequence Succ-Asp-Ser-D-Trp—N(Me)Val-βAla-Leu-Met-NH₂, SEQ ID NO. 6) were dissolved in the Tris-HCl solution. Then, each of the various solutions was mixed with 3 Kg of basic feed pellets using an electric mixer. The peptide-feed mixtures where then dried either in oven at 40° C. for 24 hours or in drying drum for few hours to form coated pellets. The feed compositions, containing different amount of peptide coating are summarized in Table 1. The total amount of each feed was 3 Kg.

TABLE 1
Compositions of feed coating and peptide doses for treatment
groups of experiments #1 and #2

| Treatment | Feed | | Final Peptide Dose |
|---|---|---|---|
| Group | type | Coating composition | (mg AN6/Kg feed) |
| A Phase 1 | Feed A | 9 ml of 50 mM Tris-HCl | 0 |
| (control) | | 51 ml distilled H2O | |
| B Phase 1 | Feed C | 3 ml of 50 mM Tris-HCl | 10 |
| | | 57 ml distilled H2O | |
| | | 30 mg AN6 | |
| A Phase 2 | Feed A | 9 ml of 50 mM Tris-HCl | 0 |
| (control) | | 51 ml distilled H2O | |
| B Phase 2 | Feed D | 9 ml of 50 mM Tris-HCl | 30 |
| | | 51 ml distilled H2O | |
| | | 90 mg AN6 | |
| C | Feed A | 9 ml of 50 mM Tris-HCl | 0 |
| (control) | | 51 ml distilled H2O | |
| D | Feed B | 3 ml of 50 mM Tris-HCl | 5 |
| | | 57 ml distilled H2O | |
| | | 15 mg AN6 | |
| E | Feed C | 3 ml of 50 mM Tris-HCl | 10 |
| | | 57 ml distilled H2O | |
| | | 30 mg AN6 | |

### Example 2. Oral Tachykinin Antagonist Treatment Improves Growth of Adult Fish

Tilapia fish (n=3600) have been raised from age 10 to 106 days post-hatch (dph) in indoor tanks kept under artificial lightening (12 h light; 12 h dark), and then transferred to cages in ponds for 160 days (total 8-9 months). The fish were fed to apparent satiation 2-4 times a day, for about 90 days (10-106 dph, Phase 1), either with regular feed (Feed A, Group A, n=600 in 3 repeats) or with feed coated with AN6 peptide solution in a ratio of 10 mg AN6/Kg feed (10 ppm, Feed C, Group B, n=600 in 3 repeats). In the second phase, fish in group B were fed for 50 days (207-257 dph, Phase 2) with feed coated with AN6 peptide solution, in a ratio of 30 mg AN6/Kg feed (30 ppm, Feed D, Group B). Between days 107 and 206 the fish received control feed (feed A). The experimental conditions are summarized in Table 2.

TABLE 2
Summary of experimental conditions and treatments (experiment #1).

| | | | Initial | Final | |
|---|---|---|---|---|---|
| | Tanks/ | | density | density | |
| Age | Cages | Tanks | (Kg | (Kg | |
| (dph) | size (L) | location | Fish/m³) | Fish/m³) | Treatment |
| 10-83 | 400 | Indoors | 0.075 | 25 | Feed A/Feed C |
| 83-106 | 200 | Indoors | 13 | 130 | Feed A/Feed C |
| 106-195 | 10400 | Outdoors | 2.5 | 9.6 | Feed A |
| 195-263 | 41600 | Outdoors | 2.3 | 4.3 | Feed A/Feed D |

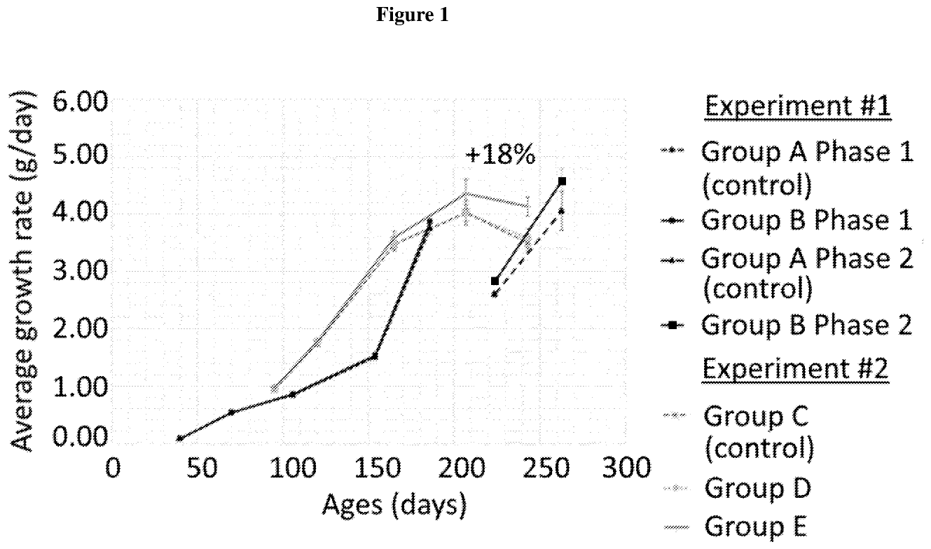

During the experiment period, the average fish weight was measured, and the daily growth rate was calculated by dividing the average weight gained in each measuring period by the number of days since the last measurement. The results showed that there were no differences in fish average weight and daily growth rate during the first 200 days of experiment (FIG. 1, experiment #1). However, at the second phase of experiment (adult fish; 195-263 dph), 50 days of feeding with AN6 (30 mg AN6/Kg feed), resulted in a significant increase in average fish weight (+6.84%) and average weight gained (+13%) compared to control fish population. This effect was also evident in the daily growth rate (FIG. 1, experiment #1). The relatively low growth rate measured in the initial phase of this experiment probably stemmed from high stocking density in tanks system in the initial period (Phase 1, Table 2, until day 106 post-hatch). This growth restriction was fully compensated in the larger cages in ponds (178 dph) where stocking density was much lower (from 106 dph, <10 Kg fish/m3).

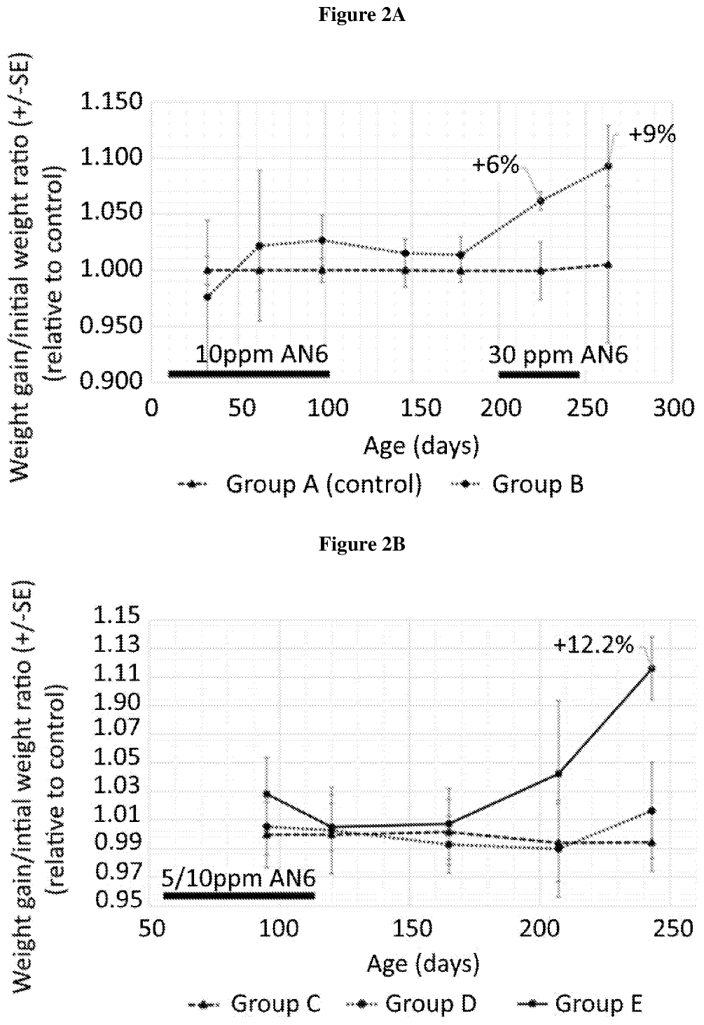

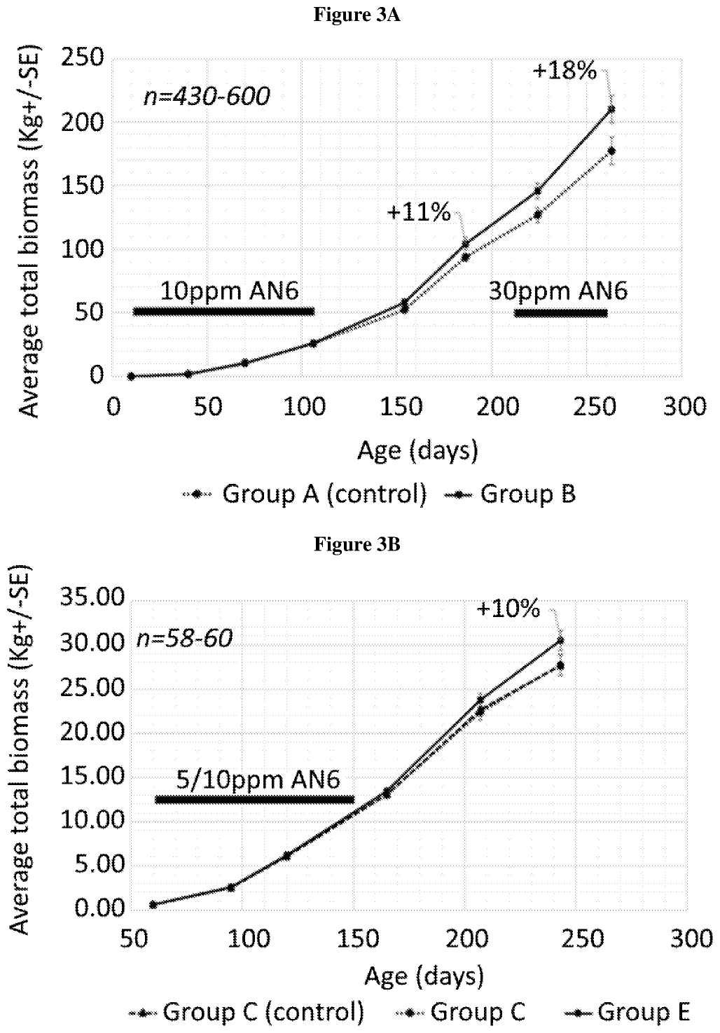

In order to eliminate the effect of initial body weight on average growth rate, the weight gained between each two measuring points was normalized according to the initial body weight, showing the same trend (FIG. 2A, 6% and 9% difference compared to the control group, at the second phase of the experiment), indicating that fish treated with AN6 grew at a significantly higher rate than expected from age 200 dph (˜6 months). The effect of AN6 treatment on fish weight was also reflected in the increased total biomass observed (+18%, FIG. 3A), which is also attributed to increased survival of fish treated with AN6. In addition, analysis of fish size distribution revealed that the number of fish in the upper 6.5th decile is higher in AN6 treatment group vs control (12% vs 6% respectively) while the number of fish in the lower 2^{th} decile is lower (7% vs 9% respectively).

### Example 3: Oral Tachykinin Antagonist Treatment Improves Growth of Adult Fish

Tilapia fish (n=1440) have been raised from day 60 to day 119 post-hatch in indoor tanks and then transferred to outdoor tanks (120-150 dph) followed by cages in ponds (150-243 dph). The fish were fed to apparent satiation 2-4 times a day, for 90 days (60-150 dph) either with regular feed (Feed A, Group C, n=60 in 8 repeats); feed coated with AN6 peptide solution in ratio of 5 mg AN6/Kg feed (5 ppm, Feed B, Group D, n=60 in 8 repeats); or feed coated with AN6 peptide solution in ratio of 10 mg AN6/Kg feed (Feed C, Group E, n=60 in 8 repeats). The experimental conditions are summarized in Table 3.

TABLE 3
Summary of experimental conditions and treatments (experiment #2).

| | | | Initial | Final | |
|---|---|---|---|---|---|
| | Tanks/ | | density | density | |
| Age | Cages | Tanks | (Kg | (Kg | |
| (dph) | size (L) | location | Fish/m³) | Fish/m³) | Treatment |
| 60-119 | 200 | Indoors | 3 | 30 | Feed A/Feed B/ |
| | | | | | Feed C |
| 120-150 | 1000 | Outdoors | 6 | 13 | Feed A/Feed B/ |
| | | | | | Feed C |
| 150-250 | 2600 | Outdoors | 5 | 8 | Feed A |

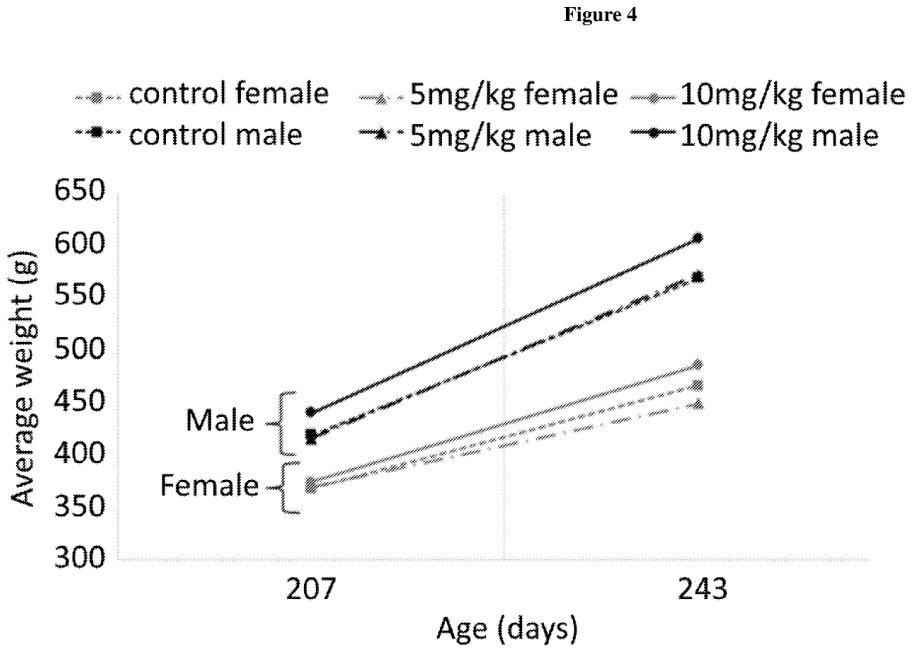

During the experimental period, the average fish weight was measured, and the daily growth rate was calculated by dividing the average weight gained in each measuring period by the number of days since the last measurement. The results showed that from age 95 to 165 dph AN6 peptide treatment (10 mg/Kg feed, Group E) resulted in steady and relatively low increase in fish average weight and growth rate (˜+3%). However, from age 200 dph, a considerable jump in fish performance and FCR was observed, reaching a maximal level at the last measurement point (243 dph, +8.4% average weight, +18% weight gain, +10% total biomass; FIG. 1, experiment #2 and FIG. 3B). The effect on growth was evident even when considering differences in initial body weight (FIG. 2B). Furthermore, analysis of weight gain of male and female populations separately revealed that both male and female had better performance than control (FIG. 4). In addition, analysis of fish size distribution revealed that the number of fish in the upper 6.5^{th} decile is higher in 10 mgAN6/Kg feed treatment (Group E) vs control (7.2% vs 2.8%, respectively) while the number of fish in the lower 2^{th} decile is lower (13.2% vs 18.4%, respectively).

The observed effect of AN6 treatment (10 mg/Kg feed) on growth rate, long after peptide treatment (age 150 dph), may point on long-term or delayed physiological changes following AN6 peptide treatment.

### Example 4: Oral Tachykinin Antagonist Treatment Improves Fish Survival

During the experiments described above, dead fish were removed from tanks before each feeding, and then counted and weighed. Dead fish were not replaced after the beginning of the experiment. In the cage systems, in which dead fish are undetectable, actual mortality was estimated by the difference between initial and final count.

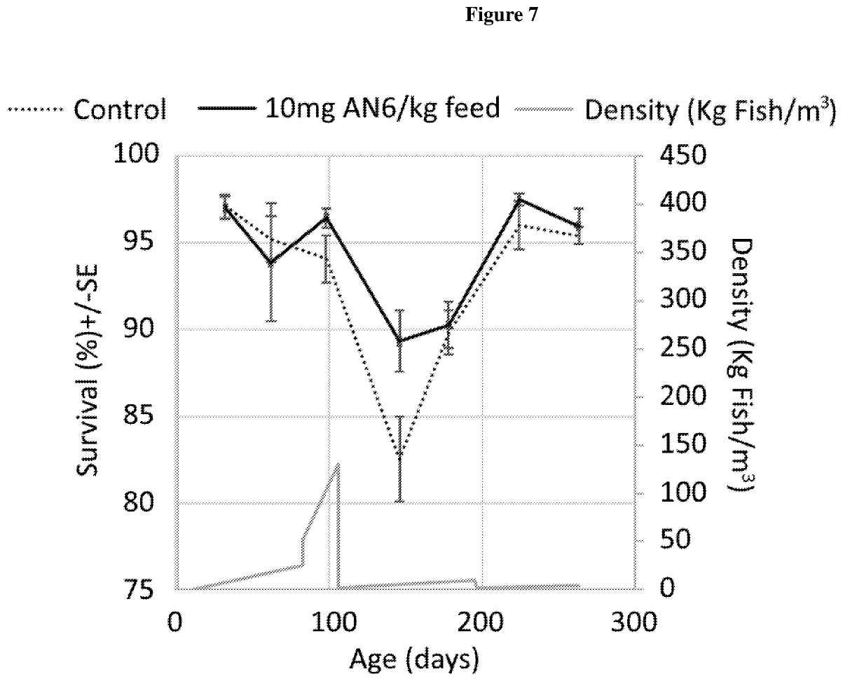

During the experiment described in example 2 (experiment #1), the fish were exposed to high stocking densities in indoor tanks (13-130 kg/m³, 83-106 dph, Table 2, FIG. 7). High stocking density is known as a major fish stressor resulting, inter alia, in chronic increase in Cortisol levels, reduced growth, changes in behavior and inhibition of immune performance. Although mortality in these tanks was not high, a significant increase in mortality was observed following fish transfer to the pond (FIG. 7, 17.5%±2.4 mortality, x̅±SE). Surprisingly, TK antagonist treatment had a significant protective effect in the pond, reducing fish mortality in 8.5% as compared to the control groups (FIG. 7, 10.5%±1.8 mortality, x̅±SE).

Pond water differ from tank water in some parameters including the presence of cellular organisms such as algae, small arthropod and bacteria that are normally absent in the relatively clean tank water. While algae and small arthropod are added directly to the fish diet, bacteria and virus can challenge fish immune system.

The importance of functional immune system in improving fish survival following transfer was suggested regarding the migration of wild salmonid smolts from their nursery lake to the north pacific ocean (Jeffries et al. Mol Ecol. 2014, 23(23), 5803-5815). This study showed that differences in immune gene expression can predict the ability of smolts to survive viral pathogens and elevated stress levels during the freshwater migration.

The high mortality observed in the pond may therefore be attributed to exposure to pathogens inside the pond, followed by death due to compromised immune system, and/or elevated stress levels during migration. It is proposed that treatment with the TK antagonist AN6 improves fish immune response, mainly in the GI tract, and reduced stress levels resulting in better survival in the pond (FIG. 7).

### Example 5: Oral Tachykinin Antagonist Treatment Improves Growth Tilapia Fish (Experiment #3)

The following fish feed was used in this experiment: MeM (powder, manufactured by Bern Aqua), Nanolis P1, P2 (pellet sizes of 1 mm, 1.2-1.5 mm, respectively, manufactured by Ocialis) and Tilaphi 2, 3 & 4 (pellet sizes of 2 mm, 3-4 mm & 5.5 mm manufactured by Ocialis). The feed content is in described in Table 4.

TABLE 4
Composition of feed coating per 3 kg of feed (for use in experiment #3)

| | | Final Peptide Dose |
|---|---|---|
| Feed type | Coating composition | (mg AN6/Kg feed) |
| Feed E (Control diet) | 30 ml distilled H2O | 0 |
| Feed F (5 ppm AN6) | 1.25 ml of 50 mM Tris-HCl | 5 |
| | 28.75 ml distilled H2O | |
| | 15 mg AN6 | |
| Feed G (10 ppm AN6) | 2.5 ml of 50 mM Tris-HCl | 10 |
| | 27.5 ml distilled H2O | |
| | 30 mg AN6 | |
| Feed H (30 ppm AN6) | 7.5 ml of 50 mM Tris-HCl | 30 |
| | 22.5 ml distilled H2O | |
| | 90 mg AN6 | |

Feed was coated using the following general procedure: Different amounts of the peptide AN6 (SEQ ID NO: 6) were prepared by dissolving the peptide in Tris-HCl solution (50 mM, pH=8.8) in ratio of 15 mg peptide to 1.25 ml Tris-HCl solution. The Tris-HCl-Peptide solution was then diluted to final volume of 30 ml with distilled H₂O as indicated in Table 4). Then, each of the various solutions was mixed with 3 Kg of the basic feed pellets (depending on fish size) using an electric mixer. The peptide-feed mixtures where then dried either in oven at 40° C. for 24 hours or in drying drum (at room temperature) for few hours to form coated pellets.

Tilapia fish (n=4800) were raised from day 10 (0.05 g) post-hatch (dph) in indoor tanks and then transferred to outdoor cages in ponds (40-279 dph). Each cage/tank is an experimental unit. The fish were fed to apparent satiation 2-4 times a day with feed appropriate to their age and treatment as described in Table 5.

TABLE 5
Feed type and feeding frequency (experiment #3)

| Stages | Ages | Pellet sizes | Frequency | Feeding time |
|---|---|---|---|---|
| 1 | 10 DOA-20 DOA | 300-500 MeM | 4 | 8:00; 10:30; 13:00; 15:00 |
| 2 | 20 DOA-30 DOA | 500-800 MeM | 4 | 8:00; 10:30; 13:00; 15:00 |
| 3 | 30 DOA-50 DOA | Nanolis 1 (1 mm) | 4 | 8:00; 10:30; 13:00; 15:00 |
| 4 | 50 DOA-80 DOA | Nanolis 2 (1.2-1.5 mm) | 4 | 8:00; 10:30; 13:00; 15:00 |
| 5 | 80 DOA-120 DOA | Tilaphi 2 (2.0 mm) | 2 | 8:00; 15:00 |
| 6 | 120 DOA-150 DOA | Tilaphi 3 (3-4 mm) | 2 | 8:00; 15:00 |
| 7 | 150 DOA-180 DOA | Tilaphi 3 (5.5 mm) | 2 | 8:00; 15:00 |
| 8 | 180 DOA-270 DOA | Tilaphi 4 | 2 | 8:00; 15:00 |

The trial included total 48 cages representing 8 treatments, with 6 repeats each. Each cage contained about 100 fish. Treatments are below:
Treatment 1 (Control): The control diet (Feed E) without the addition of the peptide (AN6). Fed to fish throughout the entire growth-trial period (for 279 days);
Treatment 2 (5 ppm from 10 dph for 120 days): The control diet with the addition of 5 mg peptide/kg of diet (Feed F). Fish were fed the peptide enriched diet for 120 days (4 months), from 10 days of age (D0—the first day of the experiment) to 130 days of age (D120), and the control diet during the last 6 months (159 days) (Feed E);
Treatment 3 (10 ppm from 10 dph for 60 days): The control diet with the addition of 10 mg peptide/kg of diet (Feed G). Fish were fed the peptide enriched diet for 60 days (2 months) from 10 days of age (D0) to 70 days of age (D60), and the control diet during the last 8 months (219 days) (Feed E);
Treatment 4 (10 ppm from 10 dph for 90 days): The control diet with the addition of 10 mg peptide/kg of diet (Feed G). Fish were fed the peptide enriched diet for 90 days (3 months) from 10 days of age (D0) to 100 days of age (D90) and the control diet for the last 7 months (189 days) (Feed E);
Treatment 5 (10 ppm from 10 dph for 120 days): The control diet with the addition of 10 mg peptide/kg of diet (Feed G). Fish were fed the peptide enriched diet for 120 days (4 months) from 10 days of age (D0) to 130 days of age (D120) and the control diet for the last 6 months (159 days) (Feed E);
Treatment 6 (10 ppm from 100 dph for 30 days): The control diet with the addition of 10 mg peptide/kg of diet (Feed G). Fish were fed the peptide enriched diet for 30 days (1 month) from 100 days of age (D90) to 130 days of age (D120) and the control diet for the first three months (90 days from D0 to D90) and the last 6 months (159 days) (Feed E);
Treatment 7 (30 ppm from 10 dph for 60 days): The control diet with the addition of 30 mg peptide/kg of diet (Feed H). Fish were fed the peptide enriched diet for 60 days (2 months) from 10 days of age (D0) to 70 days of age (D60), and the control diet during the last 8 months (219 days) (Feed E);
Treatment 8 (30 ppm from 10 dph for 90 days): The control diet with the addition of 30 mg peptide/kg of diet (Feed H). Fish were fed the peptide enriched diet for 90 days (3 months) from 10 days of age (D0) to 100 days of age (D90) and the control diet for the last 7 months (189 days) (Feed E).

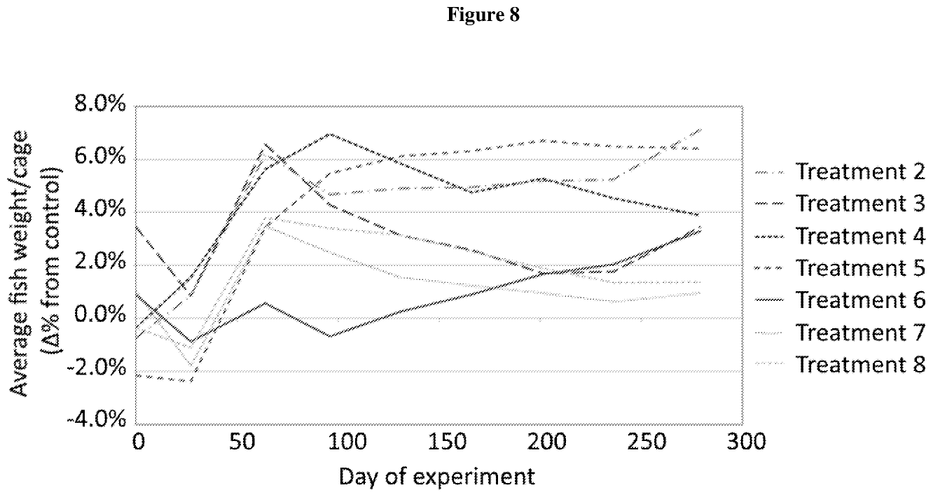

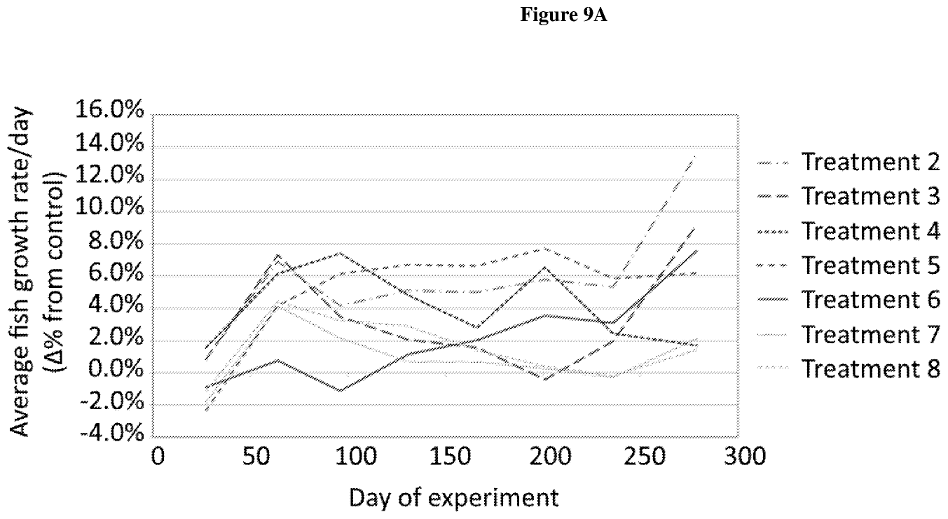

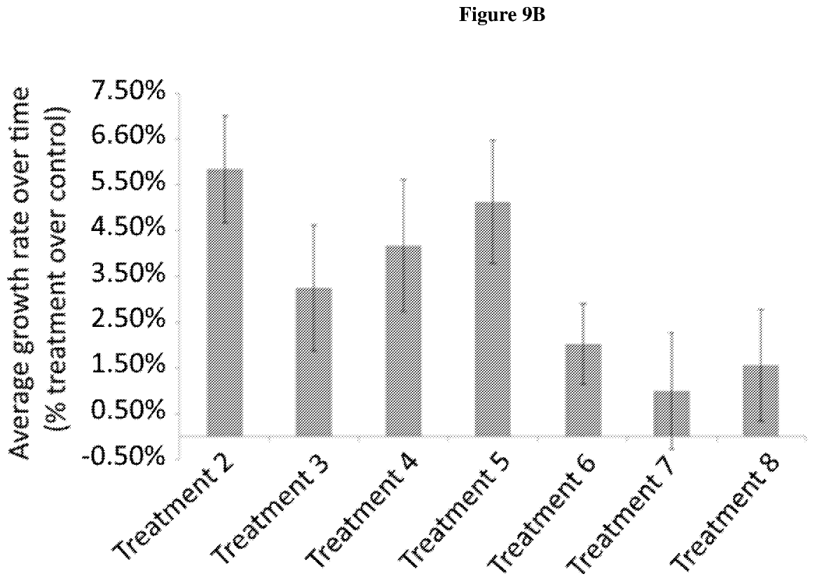

During the experimental period, fish weight and length were measured once a month. Individual fish sex was determined at days 96 and 279 of the experiment. Average fish weight per cage were calculated for each measurement period (FIG. 8). The results show that fish fed AN6-coated feed with a concentration of 10 mg AN6/Kg feed from 10 dph for 90 days (Treatment 1) or 120 days (Treatment 5) or 5 mg AN6/Kg for 120 days (Treatment 2) experience a significant increase in average fish weight (D279, +3.89%, +6.41% & +7.11% compared to control). Between two weight measurements, average weight gain (FIG. 9A) and daily growth rates (FIG. 9B) were calculated by subtracting the average weight at the beginning and end of the measuring period and dividing by the number of days between. Fish fed with 5 ppm and 10 ppm AN6 for sufficient time (>90 days) had an increased daily growth rate compared to the control that was steady throughout the experimental period (5.8% on average compared to the control) Experiment #2 and #3 were characterized by high fish survival rates (demonstrated by average fish density body/cage, total weight/cage, Table 6).

TABLE 6
Effect of TK antagonist on average survival, weight, weight
gain and FCR in last month of experiment (D 236 to D 279)

| | Average | Total | Average | | Average | Average |
|---|---|---|---|---|---|---|
| Treatment | density | weight/ | weight/ | | weight gain/ | FCR/ |
| group | (body/cage) | cage (g) | fish (g) | | fish (g) | cage |
| 2 | 0.45% | 6.71% | 6.41% | p = 0.0006 | 6.18% | −2.14% |
| 3 | 6.91% | 10.27% | 3.44% | p = 0.0776 | 9.19% | −4.28% |
| 4 | 3.34% | 7.37% | 3.89% | p = 0.0222 | 1.69% | −2.14% |
| 5 | −1.78% | 1.28% | 3.29% | | 7.59% | −0.53% |
| 6 | −1.55% | −0.53% | 0.97% | | 2.11% | 0.00% |
| 7 | −4.89% | −4.56% | 1.37% | | 1.43% | 0.00% |
| 8 | −0.67% | 6.23% | 7.11% | p = 0.0001 | 13.57% | −2.14% |

All values were calculated as % change relative to control.

Differential analysis of male vs female performances revealed that both males and females are affected by AN6 treatment, having improved average weight compared to their respective controls (Tables 7A and B).

TABLE 7A
Effect of TK antagonist on average female weight and length (experiment #3)

| | D 96 | | | | D 279 | | | |
|---|---|---|---|---|---|---|---|---|
| Female Fish | Weight/fish (g) | | Length/fish (cm) | | Weight/fish (g) | | Length/fish (cm) | |
| Treatment | Mean | Std Err | Mean | Std Err | Mean | Std Err | Mean | Std Err |
| 1 (Control) | 82.22 | 1.44 | 15.79 | 0.09 | 601.9 | 8.91 | 30.13 | 0.16 |
| 2 | 87.35 | 1.24 | 15.85 | 0.08 | 651.83 | 10.43 | 30.76 | 0.15 |
| 3 | 87.67 | 1.52 | 16.03 | 0.09 | 629.02 | 9.95 | 30.5 | 0.15 |
| 4 | 86.4 | 1.53 | 15.96 | 0.09 | 631.17 | 9.45 | 30.48 | 0.14 |
| 5 | 82.96 | 1.54 | 15.95 | 0.09 | 611.23 | 12.45 | 30.33 | 0.19 |
| 6 | 87.28 | 1.41 | 16.04 | 0.09 | 617.44 | 9.62 | 30.45 | 0.15 |
| 7 | 82.52 | 1.44 | 15.73 | 0.08 | 628.2 | 10.42 | 30.53 | 0.14 |
| 8 | 89.02 | 1.55 | 16.17 | 0.09 | 649.16 | 11.37 | 30.65 | 0.16 |

Δ Mean % (Treatment vs. control)

| | | | | | | | | |
|---|---|---|---|---|---|---|---|---|
| 2 | 6.24% | p = 0.0090 | 0.38% | | 8.30% | p = 0.0004 | 2.09% | p = 0.0027 |
| 3 | 6.63% | p = 0.0079 | 1.52% | p = 0.0433 | 4.51% | | 1.23% | |
| 4 | 5.08% | p = 0.0418 | 1.08% | | 4.86% | p = 0.0418 | 1.16% | |
| 5 | 0.90% | | 1.01% | | 1.55% | | 0.66% | |
| 6 | 6.15% | p = 0.0136 | 1.58% | p = 0.0391 | 2.58% | | 1.06% | |
| 7 | 0.36% | | −0.38% | | 4.37% | | 1.33% | |
| 8 | 8.27% | p = 0.0010 | 2.41% | p = 0.0016 | 7.85% | p = 0.0010 | 1.73% | |

TABLE 7B
Effect of TK antagonist on average male weight and length (experiment #3)

| | D 96 | | | | D 279 | | | |
|---|---|---|---|---|---|---|---|---|
| Male Fish | Weight/fish (g) | | Length/fish (cm) | | Weight/fish (g) | | Length/fish (cm) | |
| Treatment | Mean | Std Err | Mean | Std Err | Mean | Std Err | Mean | Std Err |
| 1 (Control) | 105.5 | 1.29 | 17.22 | 0.07 | 865.31 | 10.21 | 34.38 | 0.13 |
| 2 | 111.8 | 1.44 | 17.22 | 0.08 | 916.05 | 11.92 | 34.37 | 0.15 |
| 3 | 106.51 | 1.26 | 17.19 | 0.07 | 877.38 | 11.21 | 34.14 | 0.13 |
| 4 | 111.87 | 1.38 | 17.26 | 0.07 | 881.92 | 10.87 | 34.15 | 0.12 |
| 5 | 100.46 | 1.12 | 17.08 | 0.06 | 859.1 | 10.16 | 34.21 | 0.12 |
| 6 | 105.78 | 1.33 | 17.07 | 0.07 | 863.26 | 11.6 | 34.19 | 0.13 |
| 7 | 109.68 | 1.54 | 17.78 | 0.62 | 840.39 | 9.84 | 34.04 | 0.13 |
| 8 | 106.64 | 1.26 | 17.21 | 0.07 | 912.05 | 11.13 | 34.56 | 0.12 |

Δ Mean % (Treatment vs. control)

| | | | | | | |
|---|---|---|---|---|---|---|
| 2 | 5.97% | p = 0.0011 | 0.00% | 5.86% | p = 0.0013 | −0.03% |
| 3 | 0.96% | −0.17% | | 1.39% | | −0.70% |
| 4 | 6.04% | p = 0.0006 | 0.23% | 1.92% | | −0.67% |
| 5 | −4.78% | p = 0.0060 | −0.81% | −0.72% | | −0.49% |
| 6 | 0.27% | −0.87% | | −0.24% | | −0.55% |
| 7 | 3.96% | p = 0.0289 | 3.25% | −2.88% | | −0.99% |
| 8 | 1.08% | | −0.06% | 5.40% | p = 0.0028 | 0.52% |

Fish size distributions reveals a higher proportion of large fish (>1000 g) and a lower proportion of small fish (<476 g) in AN6 treated groups (Treatments 2, 3, or 8) (Table 8).

TABLE 8
Effect of TK antagonist on total number of large and small fish in each treatment

| | Big fish 279 dph >1000 g | | | Small fish 279 dph >476 g | | |
|---|---|---|---|---|---|---|
| Treatment | Fish# | Big fish # | % big fish | Fish # | Small fish # | % small fish |
| Control | 449 | 46 | 10.2% | 449 | 41 | 9.1% |
| 2 | 451 | 75 | 16.6% | 451 | 21 | 4.7% |
| 3 | 480 | 79 | 16.5% | 480 | 23 | 4.8% |
| 4 | 463 | 68 | 14.7% | 463 | 22 | 4.8% |
| 5 | 441 | 53 | 12.0% | 441 | 38 | 8.6% |
| 6 | 442 | 57 | 12.9% | 442 | 29 | 6.6% |
| 7 | 427 | 46 | 10.8% | 427 | 23 | 5.4% |
| 8 | 446 | 91 | 20.4% | 446 | 25 | 5.6% |

Δ Treatment - Control (%)

| | | | | |
|---|---|---|---|---|
| 2 | 6.4% | p = 0.0397 | −4.5% | p = 0.0139 |
| 3 | 6.2% | p = 0.0459 | −4.3% | p = 0.0164 |
| 4 | 4.4% | | −4.4% | p = 0.0187 |
| 5 | 1.8% | | −0.5% | |
| 6 | 2.7% | | −2.6% | |
| 7 | 0.5% | | −3.7% | p = 0.0267 |
| 8 | 10.2% | p = 0.0019 | −3.5% | p = 0.0395 |

The observed effect of AN6 treatment (5 and 10 mg/Kg feed) on average weight and growth rate, is significant 159 to 189 days after peptide treatment ended, may point on long-term or delayed physiological changes following AN6 peptide treatment.

### Example 6: Oral Tachykinin Antagonist Treatment Improves Feed Conversion Rate (FCR) in Adult Fish

In order to determine the efficiency of feed conversion into fish substance in experiments (#1, #2, #3), FCR values which reflect the amount of feed needed to grow a kilogram fish, were calculated for each period and for the entire fish growth period. In order to determine the amount of consumed feed, the amount of feed provided was measured daily for each tank and the leftovers were collected, weighted and subtracted from the total amount of feed. Dead fish were removed every day before each feeding and then counted and weighed for accurate calculation of feed intake and FCR. It is to be noted that high mortality can still influence FCR values since feed eaten by fish that have later died cannot be subtracted from the total feed consumed and on the other hand periodic total fish weight gained is underestimated (dead fish are not included). Three FCR values were calculated according to the following formulas:

$${FCR}_{a} = \frac{total amount of feed consumed / final number of fish}{initial average weight - final average weight}$$

$${FCR}_{b} = \frac{ \begin{matrix}Total amount of feed consumed \\ between to measurment points\end{matrix} }{Total weight gained between to measurment points}$$

$${FCR}_{c} = \frac{ \begin{matrix}Total amount of feed \\ consumed for entire fish growth period\end{matrix} }{Final average fish weight}$$

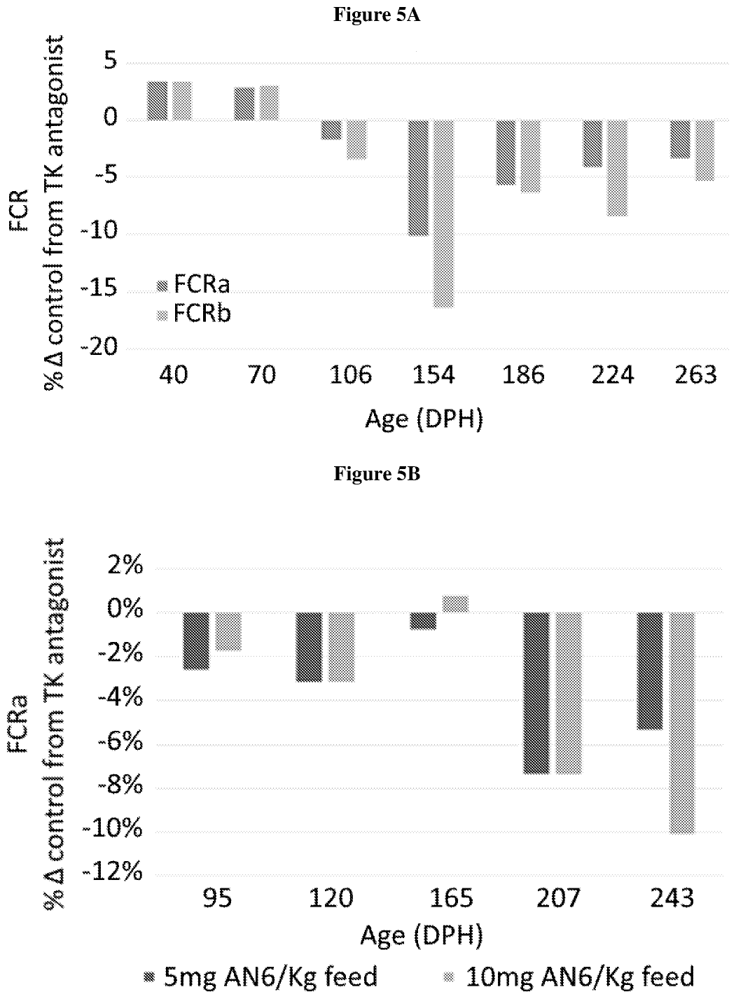

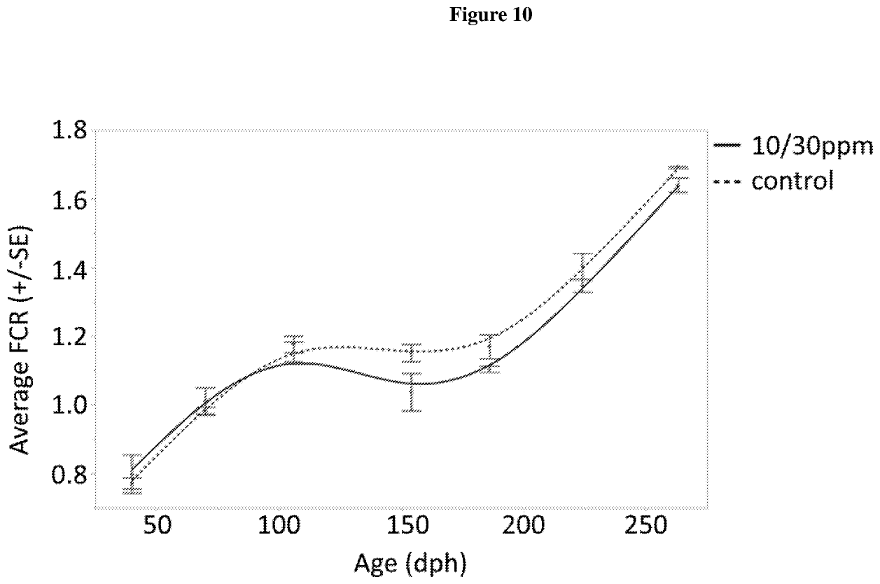

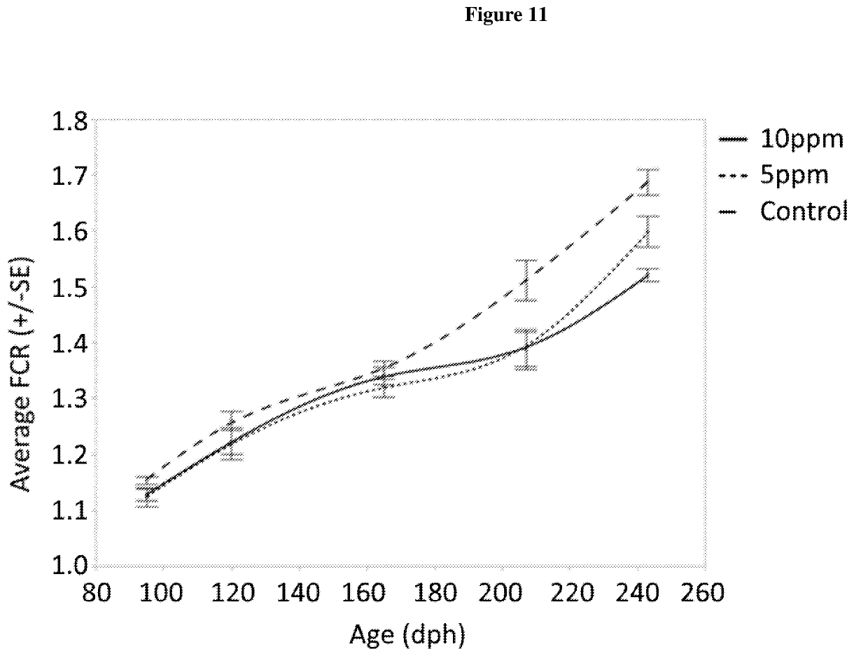

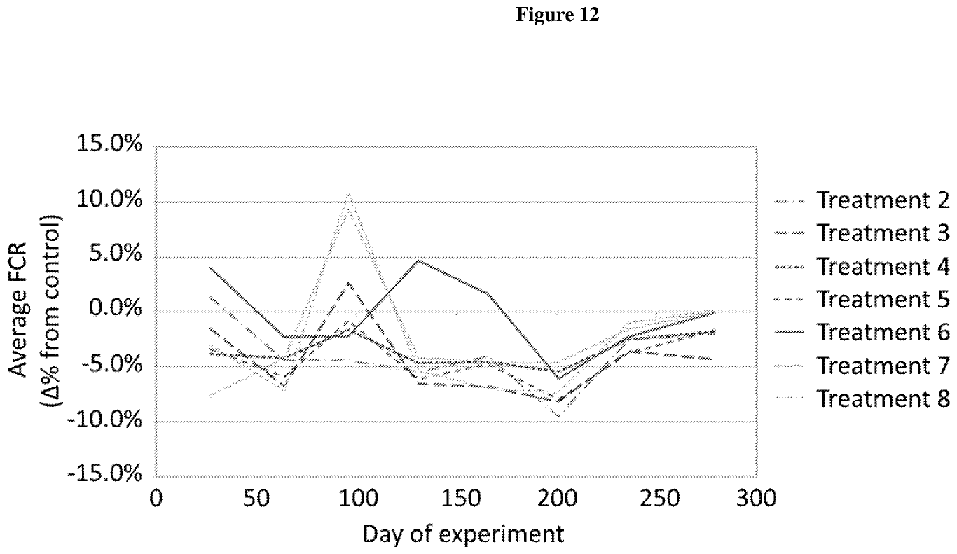

The calculated FCR values clearly indicate that all AN6 peptide treatments significantly improves FCR in adult fish (FIGS. 5A, 5B, 10, 11, 12). Specifically, improved FCR is observed 30 to 90 days after treatment initiation and can last at least 100 days after treatment stopped. Treatment with 5 mgAN6/Kg feed from 60 dph for 150 days was not sufficient to improved growth but improved FCR significantly (experiment #2). Similarly, in experiment #3, AN6 treatment for 60 days in concentration of 10 mg/Kg feed (Treatment 3) and AN6 treatment for 90 days in concentration of 30 mg/Kg feed (Treatment 8) did not affect growth but improved FCR significantly. Improved FCR is also observed in the period after AN6 peptide treatment (FIGS. 10, 11, 12), suggesting an anatomical and/or physiological change in the fish.

The physiology of improved FCR can be broken into several successive steps: Feed Intake→Digestion→Absorption→Conversion. While digestion and absorption relate to gut physiology, conversion occurs mainly in the soma and is determined by the availability of metabolites and energy in growth processes on the expanse of other energy consuming processes (metabolism).

In the relationship between FCR and growth, two main dogmas are possible: a. Improved FCR value which is a manifestation of similar growth performance with less feed consumed. b. Improved FCR that occurs when fish have better growth with similar or higher feed intake. Namely, improved FCR does not necessarily result in an increased growth rate, and vice versa—increased growth rate does not necessarily indicate an improved FCR. According to the first dogma, feed is digested and absorbed in higher efficiency, but growth potential is not altered. In the second dogma, a shift in metabolism towards growth may also be involved. Consistent with these scenarios, the current experiments showed different FCR-growth relationship. Treatment with 10 and 30 mg AN6/Kg feed resulted in both improved FCR and growth performance (experiment #1). On the other hand, treatment with 5, 10 mg and 30 mg AN6/Kg feed in other cases resulted in improved FCR which did not coincide with improved growth but rather with decrease in feed intake (experiments #2 and #3). These results suggest that TK antagonist act on gut physiology and metabolism in separate pathways. The differential activation of these pathways may correlate with treatment age and peptide dose per Kg fish at the time of treatment.

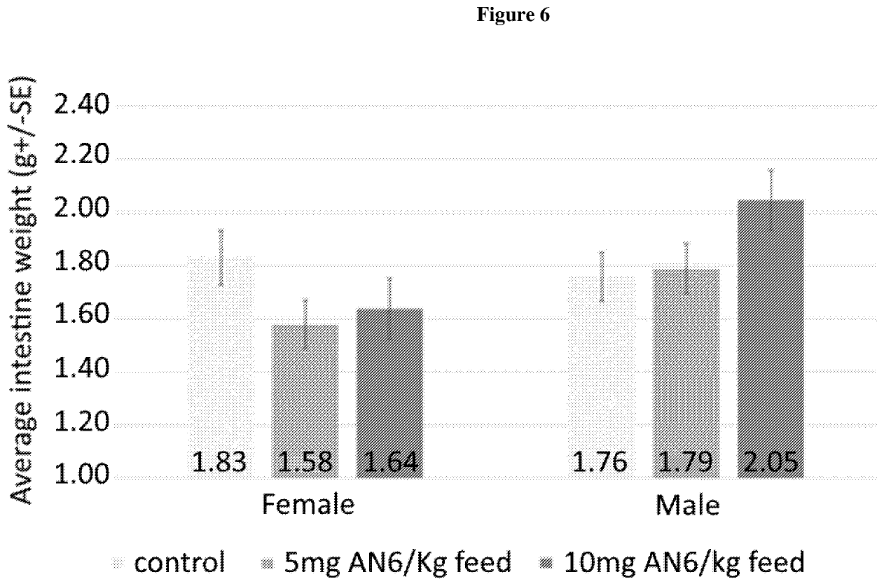

At the end of the experiment described in example 3 (experiment #2), 6 fish from each cage/tank (3 females and 3 males, n=24 per treatment group) were sacrificed and whole intestine was removed and weighted. A difference in the intestine size was observed in male fish treated with 10 mg AN6/Kg feed and the ratio between intestine weight and fish weight (ISI) was significantly higher in male treated with 10 mg AN6/Kg feed (+21%, FIG. 6). These results indicate that AN6 peptide treatment have the capacity to affect the physiology and/or anatomy of the fish intestine.

### Example 7: Safety of Treatment with AN6

At the end of each of the experiments described in examples 2, 3 and 5 (#1, #2, #3), a sample of fish from each treatment group was sacrificed and dissected for measurements of internal parts as indicated in Table 9.

TABLE 9
Sacrificed samples from experiments

| | | | Average | Number | |
|---|---|---|---|---|---|
| | | | fish | of fish | Total |
| | | Cage | number/ | sampled/ | fish |
| Experiment | Treatment | number | cage | cage | sampled |
| 1 | 2 | 6 | 360 | 5 Males & | 60 |
| | | | | 5 Females | |
| 2 | 3 | 24 | 53 | 3 Males & | 144 |
| | | | | 3 Females | |
| 3 | 8 | 48 | 73 | 3 | 144 |

No significant effect of AN6 treatment on gonad size and histology was observed in any of the experiments. Similarly, no effect of treatment with AN6 was observed on kidney, liver and fat weight and rates in any of the experiments.

It was also demonstrated that the half-life time of AN6 in blood is very short and that non residual peptide was observed in muscle and plasma following treatment, indicating the safety of the compound.

Fillet rate (calculated as the ratio between the maximal boneless longitudinal fish muscle slice, from head to tail, and total fish mass), was significantly higher in males and females treated with 10 ppm AN6 (experiments 2 & 3, +3.38% female fillet ratio, +5.6% male fillet ratio).

### Example 8: In Vitro Inhibition of Tilapia and Salmon NKB Receptor Expressed in Naïve COS7 Cells

Naïve COS7 cells (fibroblast-like cell lines derived from monkey kidney tissue) were transfected with DNA of Tilapia Tac3Ra (t1NKB receptor a, cloned in pcDNA3.1). Exogenous receptor activity is determined by dynamic reads of Fluorescent emission from Ca++ indicator reporter system (Fluo4AM, Molecular Probes). Fluo-4 molecule and their derivatives all exhibit large increase in fluorescence emission intensity upon Ca++ binding. Time-laps reading of florescence intensity initiates ˜12 sec after activator is added to the cells, in 6 sec intervals for 2 minutes (readmax˜30-40 sec). Alternatively, Naïve COS7 cells were co-transfected with DNA of Salmon Tac3R (NKB receptor, slNKBr Cloned in pcDNA3.1) and Luciferase reporter under the regulation of SRE promoter. Nuclear SRE promoter activation is part of the PKA signaling pathway that is typical for NKB receptor activation. In this case, salmon NKB receptor activity is determined by reading of Luciferase fluorescence emission in plate reader 6 hours after activation.

For background subtraction, average reads in the last minutes is subtracted from reads in the first minutes which includes peak Ca++ fluorescence activity (Afluorescence). Read over time are smoothen by Polynomial Fit Degree=4 (JMP®, SAS). Peak value (Apeak fluorescence), usually achieved 12-24 seconds after reading initiation, is used as the result value for each concentration. For verifying ligand specific receptor activation, in each experimental setup cell activity following DMSO treatment is also determined (null). Receptor activation state in different ligand concentrations is expressed as a single peak value (Apeak fluorescence) divided by the read of maximal receptor activity concentration ((Apeak/Amax=1). EC50 is determined as AN6/Senktide concentration required for half maximal activity. IC50 is determined as AN6 concentration required for half maximal inhibition. Peptide concentration of EC50 and IC50 are predicted by Logistic 4P Roadbard Fit or Logistic 4P Fit (JMP®, SAS).

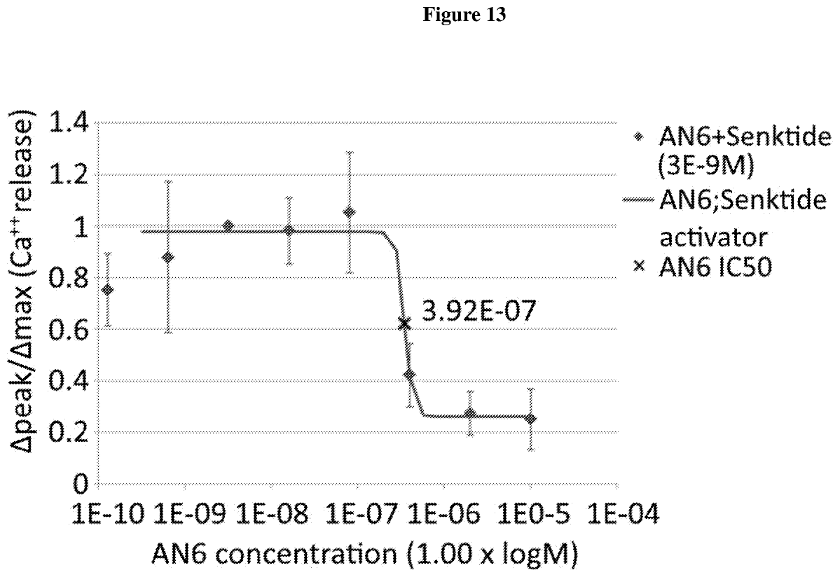

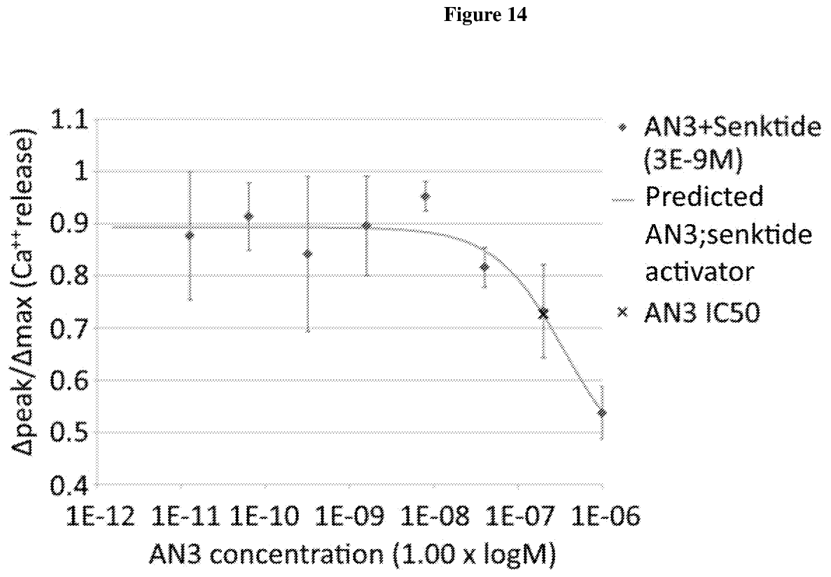

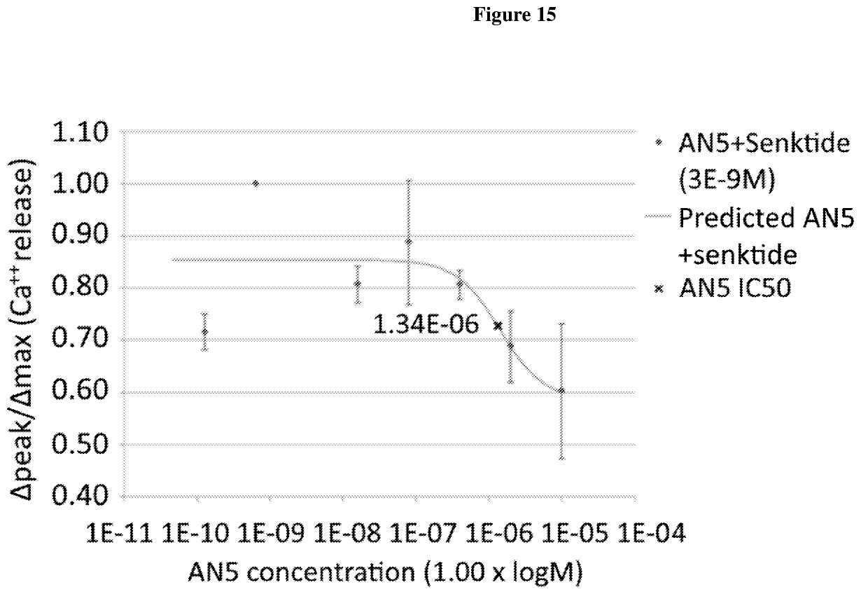

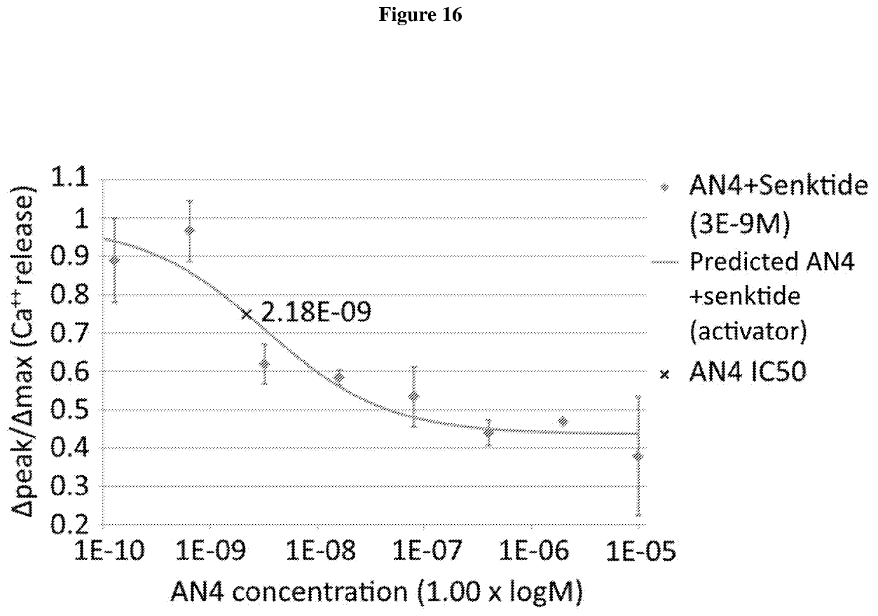

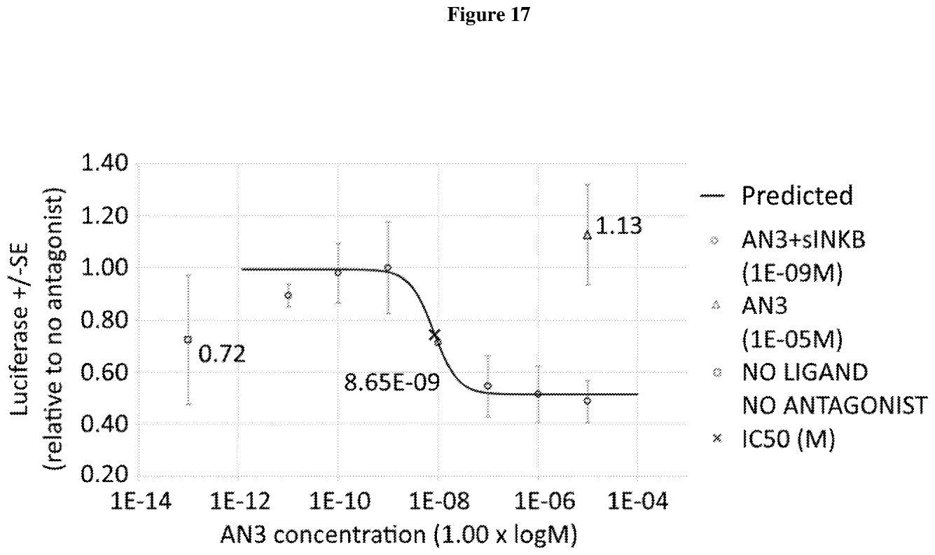

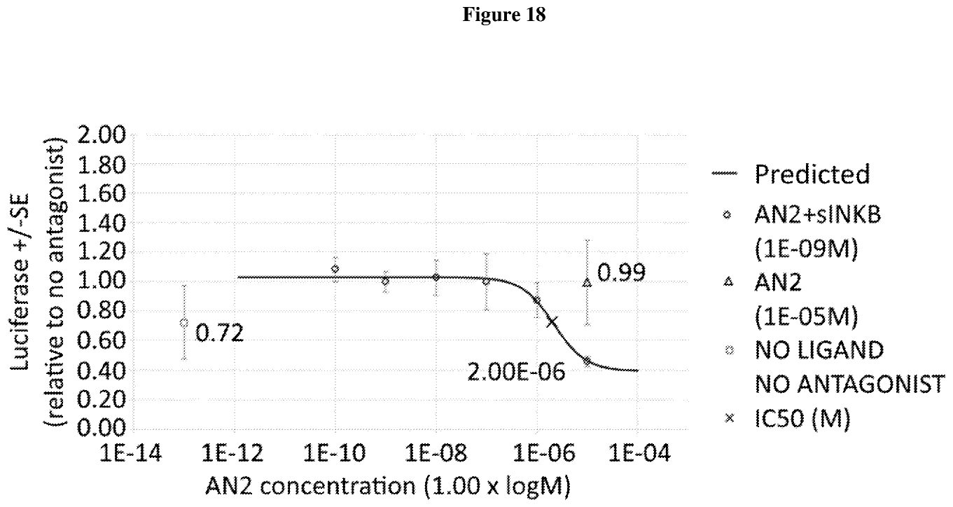

In order to determine inhibition capacity of the peptides, cells were treated with increasing concentrations of the peptide AN6, AN2, AN3, AN4 or AN5 (SEQ ID Nos: 6, 2, 3, 4 and 5 respectively), and tilapia NKBr or salmon NKBr were simultaneously activated by Senktide (Cayman), a potent, synthetic and specific NKB activator or by Salmon NKB (s1NKB) ligand, respectively (FIGS. 13 to 19).

The peptides' inhibition affinity (IC50), determined as the concentration required to reduce by half the average florescence emission (from Ca++ or luciferase reporter systems), induced by agonist in constant concentration.

It is demonstrated that all tested TK antagonists inhibit binding of salmon and tilapia NKB receptor agonist.

1-40. (canceled)

41. A method of improving fish feed conversion rate (FCR), the method comprising administering to a fish population a composition comprising at least one tachykinin (TK) antagonist capable of binding to a piscine TK receptor and inhibiting its activity.

42. The method according to claim 41, wherein the reproductive system of the treated fish is not affected by the TK antagonist treatment.

43. The method according to claim 41, wherein the TK antagonist is a peptide-based molecule or a peptidomimetic.

44. The method according to claim 43, wherein the peptidomimetic is according to Formula I:
X₁—NMeVal-X₄-Leu-Met-Z (Formula I),
wherein:
the peptidomimetic consists of 5-10 amino acids;
X₁ is a stretch of 1-6 natural or non-natural amino acid residues and optionally an N-terminal capping moiety or modification;
NMeVal is an N-methyl-Valine residue or N-methyl-D-Valine residue;
X₄ is —NH(CH₂)ₙ—CO— wherein n is 2-6; and
Z is the C-terminus of the peptidomimetic which may be amidated, acylated, reduced, or esterified.

45. The method according to claim 44, wherein X₁ consists of 2 or 3 amino acid residues comprising an aromatic residue, a negatively charged residue and an N-terminus capping moiety.

46. The method according to claim 44, wherein the N-terminal capping moiety is a dicarboxylic acid residue.

47. The method according to claim 44, wherein the peptidomimetic consists of 5-10 amino acid residues comprising the sequence NMeVal-βAla-Leu-Met (SEQ ID NO. 7), and a sequence selected from the group consisting of: Succ-Asp-Phe; Succ-Asp-DPhe; Succ-Asp-Trp; Succ-Asp-DTrp; Succ-Asp-Ile-Phe; Succ-Asp-Ile-DPhe; Succ-Asp-Ile-Trp; Succ-Asp-Ile-DTrp; Succ-Asp-Ser-Phe; Succ-Asp-Ser-DPhe; Succ-Asp-Ser-Trp; Succ-Asp-Ser-DTrp, Succ-Glu-Phe; Succ-Glu-DPhe; Succ-Glu-Trp; Succ-Glu-DTrp; Succ-Glu-Ile-Phe; Succ-Glu-Ile-DPhe; Succ-Glu-Ile-Trp; Succ-Glu-Ile-DTrp; Succ-Glu-Ser-Phe; Succ-Glu-Ser-DPhe; Succ-Glu-Ser-Trp; and Succ-Glu-Ser-DTrp.

48. The method according to claim 44 wherein the peptidomimetic is selected from the group consisting of:
(SEQ ID NO. 1, AN1)
Succ-Asp-Ile-Phe-N(Me)Val-βAla-Leu-Met-NH₂;
(SEQ ID NO. 2, AN2)
Succ-Asp-Phe-N(Me)Val-βAla-Leu-Met-NH₂;
(SEQ ID NO. 3, AN3)
Succ-Asp-Ser-Phe-N(Me)Val-βAla-Leu-Met-NH₂;
(SEQ ID NO. 4, AN4)
Succ-Asp-Ile-D-Trp-N(Me)Val-βAla-Leu-Met-NH₂;
(SEQ ID NO. 5, AN5)
Succ-Asp-D-Trp-N(Me)Val-βAla-Leu-Met-NH₂;
and
(SEQ ID NO. 6, AN6)
Succ-Asp-Ser- D-Trp -N(Me)Val-βAla-Leu-Met-NH₂;
wherein Succ denotes a succinyl.

49. The method according to any one of claim 41, wherein the fish population comprises majority of fish aged 3-1200 dph.

50. The method according to claim 41, wherein the fish are edible fish.

51. The method according to claim 50, wherein the fish type is selected from the group consisting of: tilapia, carp, salmon, bass, catfish, and mullet.

52. The method according to claim 41, wherein the administering route is selected from parenteral administration, enteral administration, and administration by immersion.

53. The method according to claim 41, wherein the composition is administered to fish in a volume of water to be taken up by the gills or to be absorbed by the skin.

54. The method according to claim 53, wherein the composition is administered to fish as part of regular feed or water consumption.

55. The method according to claim 54, wherein the composition is administered as coating of feed pellets.

56. The method according to claim 55, wherein the feed pellets are coated with a final TK antagonist dose of 1-50 mg per one kg of feed.

57. The method according to claim 41, wherein the composition further comprises at least one nutrient or feed additive selected from the group consisting of color additive, taste additive, protein, carbohydrate, fat, mineral, and vitamin.

58. A method of improving at least one fish health parameter selected from the group consisting of: survival, skin condition, and resistance to pathogens; the method comprising administering to a fish population a composition comprising at least one tachykinin (TK) antagonist capable of binding to a piscine TK receptor and inhibiting its activity, wherein the TK antagonist is a peptide-based molecule or a peptidomimetic.

59. The method according to claim 58, wherein the peptidomimetic is according to Formula I:
X₁—NMeVal-X₄-Leu-Met-Z (Formula I),
wherein:
the peptidomimetic consists of 5-10 amino acids;
X₁ is a stretch of 1-6 natural or non-natural amino acid residues and optionally an N-terminal capping moiety or modification;
NMeVal is an N-methyl-Valine residue or N-methyl-D-Valine residue;
X₄ is —NH(CH₂)ₙ—CO— wherein n is 2-6; and
Z is the C-terminus of the peptidomimetic which may be amidated, acylated, reduced, or esterified.

60. The method according to claim 58 wherein the peptidomimetic is selected from the group consisting of:
(SEQ ID NO. 1, AN1)
Succ-Asp-Ile-Phe-N(Me)Val-βAla-Leu-Met-NH₂;
(SEQ ID NO. 2, AN2)
Succ-Asp-Phe-N(Me)Val-βAla-Leu-Met-NH₂;
(SEQ ID NO. 3, AN3)
Succ-Asp-Ser-Phe-N(Me)Val-βAla-Leu-Met-NH₂;
(SEQ ID NO. 4, AN4)
Succ-Asp-Ile-D-Trp-N(Me)Val-βAla-Leu-Met-NH₂;
(SEQ ID NO. 5, AN5)
Succ-Asp-D-Trp-N(Me)Val-βAla-Leu-Met-NH₂;
and
(SEQ ID NO. 6, AN6)
Succ-Asp-Ser- D-Trp -N(Me)Val-βAla-Leu-Met-NH₂;
wherein Succ denotes a succinyl.


SEQUENCE LISTING (7 sequences)

SEQ ID NO: 1 - protein, 7 aa, Artificial Sequence
  Synthetic peptide
  MOD_RES (1)..(1): ACETYLATION with Succinyl
  MOD_RES (4)..(4): MeVal
  MOD_RES (5)..(5): bAla
  MOD_RES (7)..(7): AMIDATION
DIFXXLM

SEQ ID NO: 2 - protein, 6 aa, Artificial Sequence
  Synthetic peptide
  MOD_RES (1)..(1): ACETYLATION with Succinyl
  MOD_RES (3)..(3): MeVal
  MOD_RES (4)..(4): bAla
  MOD_RES (6)..(6): AMIDATION
DFXXLM

SEQ ID NO: 3 - protein, 7 aa, Artificial Sequence
  Synthetic peptide
  MOD_RES (1)..(1): ACETYLATION with Succinyl
  MOD_RES (4)..(4): MeVal
  MOD_RES (5)..(5): bAla
  MOD_RES (7)..(7): AMIDATION
DSFXXLM

SEQ ID NO: 4 - protein, 7 aa, Artificial Sequence
  Synthetic peptide
  MOD_RES (1)..(1): ACETYLATION with Succinyl
  MOD_RES (3)..(3): D-Trp
  MOD_RES (4)..(4): MeVal
  MOD_RES (5)..(5): bAla
  MOD_RES (7)..(7): AMIDATION
DIWXXLM

SEQ ID NO: 5 - protein, 6 aa, Artificial Sequence
  Synthetic peptide
  MOD_RES (1)..(1): ACETYLATION with Succinyl
  MOD_RES (2)..(2): D-Trp
  MOD_RES (3)..(3): MeVal
  MOD_RES (4)..(4): bAla
  MOD_RES (6)..(6): AMIDATION
DWXXLM

SEQ ID NO: 6 - protein, 7 aa, Artificial Sequence
  Synthetic peptide
  MOD_RES (1)..(1): ACETYLATION with Succinyl
  MOD_RES (3)..(3): D-Trp
  MOD_RES (4)..(4): MeVal
  MOD_RES (5)..(5): bAla
  MOD_RES (7)..(7): AMIDATION
DSWXXLM

SEQ ID NO: 7 - protein, 4 aa, Artificial Sequence
  Synthetic peptide
  MOD_RES (1)..(1): MeVal
  MOD_RES (2)..(2): bAla
XXLM